(19) United States
(12) Patent Application Publication
(10) Pub. No.: US 2006/0116531 A1
(43) Pub. Date: Jun. 1, 2006

(54) Modeling of liquid-phase oxidation

(76) Inventors: Alan George Wonders, Kingsport, TN (US); Wayne Scott Strasser, Kingsport, TN (US); Puneet Gupta, Saint Peters, MO (US); Lee Reynolds Partin, Kingsport, TN (US); Marcel de Vreede, Barendrecht (NL)

Correspondence Address: Michael K. Carrier;Eastman Chemical Company, P.O. Box 511, Kingsport, TN, 37662-5075, US

(21) Appl. No.: 11/271,308

(22) Filed: Nov. 10, 2005

Related U.S. Application Data
(60) Provisional application No. 60/594,774, filed on May 5, 2005.
(60) Provisional application No. 60/631,350, filed on Nov. 29, 2004.

(51) Int. Cl. C07C 51/255; G06F 19/00

(52) U.S. Cl. 562/412; 702/22

(57) ABSTRACT

Disclosed is an optimized process for more effectively and efficiently modeling liquid-phase oxidation in a bubble column reactor.

### RELATED APPLICATION

This application claims the priority benefit of U.S. Provisional Patent Application Ser. No. 60/594,774, filed May 5, 2005 and U.S. Provisional Patent Application Ser. No. 60/631,350, filed Nov. 29, 2004, the entire disclosures of which are incorporated herein by reference.

### FIELD OF THE INVENTION

This invention relates generally to the liquid-phase, catalytic oxidation of an aromatic compound. One aspect of the invention concerns the partial oxidation of a dialkyl aromatic compound (e.g., para-xylene) in a bubble column reactor to produce a crude aromatic dicarboxylic acid (e.g., crude terephthalic acid), which can thereafter be subjected to purification and separation. Another aspect of the invention concerns a method of modeling a bubble column reactor that more accurately predicts the behavior of an actual bubble column reactor.

### BACKGROUND OF THE INVENTION

Liquid-phase oxidation reactions are employed in a variety of existing commercial processes. For example, liquid-phase oxidation is currently used for the oxidation of aldehydes to acids (e.g., propionaldehyde to propionic acid), the oxidation of cyclohexane to adipic acid, and the oxidation of alkyl aromatics to alcohols, acids, or diacids. A particularly significant commercial oxidation process in the latter category (oxidation of alkyl aromatics) is the liquid-phase catalytic partial oxidation of para-xylene to terephthalic acid. Terephthalic acid is an important compound with a variety of applications. The primary use of terephthalic acid is as a feedstock in the production of polyethylene terephthalate (PET). PET is a well-known plastic used in great quantities around the world to make products such as bottles, fibers, and packaging.

In a typical liquid-phase oxidation process, including partial oxidation of para-xylene to terephthalic acid, a liquid-phase feed stream and a gas-phase oxidant stream are introduced into a reactor and form a multi-phase reaction medium in the reactor. The liquid-phase feed stream introduced into the reactor contains at least one oxidizable organic compound (e.g., para-xylene), while the gas-phase oxidant stream contains molecular oxygen. At least a portion of the molecular oxygen introduced into the reactor as a gas dissolves into the liquid phase of the reaction medium to provide oxygen availability for the liquid-phase reaction. If the liquid phase of the multi-phase reaction medium contains an insufficient concentration of molecular oxygen (i.e., if certain portions of the reaction medium are “oxygen-starved”), undesirable side-reactions can generate impurities and/or the intended reactions can be retarded in rate. If the liquid phase of the reaction medium contains too little of the oxidizable compound, the rate of reaction may be undesirably slow. Further, if the liquid phase of the reaction medium contains an excess concentration of the oxidizable compound, additional undesirable side-reactions can generate impurities.

Conventional liquid-phase oxidation reactors are equipped with agitation means for mixing the multi-phase reaction medium contained therein. Agitation of the reaction medium is supplied in an effort to promote dissolution of molecular oxygen into the liquid phase of the reaction medium, maintain relatively uniform concentrations of dissolved oxygen in the liquid phase of the reaction medium, and maintain relatively uniform concentrations of the oxidizable organic compound in the liquid phase of the reaction medium.

Agitation of the reaction medium undergoing liquid-phase oxidation is frequently provided by mechanical agitation means in vessels such as, for example, continuous stirred tank reactors (CSTRs). Although CSTRs can provide thorough mixing of the reaction medium, CSTRs have a number of drawbacks. For example, CSTRs have a relatively high capital cost due to their requirement for expensive motors, fluid-sealed bearings and drive shafts, and/or complex stirring mechanisms. Further, the rotating and/or oscillating mechanical components of conventional CSTRs require regular maintenance. The labor and shutdown time associated with such maintenance adds to the operating cost of CSTRs. However, even with regular maintenance, the mechanical agitation systems employed in CSTRs are prone to mechanical failure and may require replacement over relatively short periods of time.

Bubble column reactors provide an attractive alternative to CSTRs and other mechanically agitated oxidation reactors. Bubble column reactors provide agitation of the reaction medium without requiring expensive and unreliable mechanical equipment. Bubble column reactors typically include an elongated upright reaction zone within which the reaction medium is contained. Agitation of the reaction medium in the reaction zone is provided primarily by the natural buoyancy of gas bubbles rising through the liquid phase of the reaction medium. This natural-buoyancy agitation provided in bubble column reactors reduces capital and maintenance costs relative to mechanically agitated reactors. Further, the substantial absence of moving mechanical parts associated with bubble column reactors provides an oxidation system that is less prone to mechanical failure than mechanically agitated reactors.

When liquid-phase partial oxidation of para-xylene is carried out in a conventional oxidation reactor (CSTR or bubble column), the product withdrawn from the reactor is typically a slurry comprising crude terephthalic acid (CTA) and a mother liquor. CTA contains relatively high levels of impurities (e.g., 4-carboxybenzaldehyde, para-toluic acid, fluorenones, and other color bodies) that render it unsuitable as a feedstock for the production of PET. Thus, the CTA produced in conventional oxidation reactors is typically subjected to a purification process that converts the CTA into purified terephthalic acid (PTA) suitable for making PET.

It is, of course, desirable to minimize the amount of impurities in the slurry produced from a bubble column reactor. However, in order to minimize impurities, the physical and chemical dynamics of the multi-phase reaction medium contained in the bubble column reactor must be understood. Because the flow fields of the multi-phase reaction medium in bubble column are quite stochastic, understanding the physical and chemical dynamics in a bubble column is not a simple matter.

Certain physical properties of the reaction medium can be measured at different locations in the reactor using non-invasive measurement techniques (e.g., radiation emission-detection). However, in order to accurately measure most physical and chemical properties throughout of the reaction medium, actual sampling of the reaction medium at a multitude of locations would be required. Obtaining enough samples throughout the reaction medium to provide and accurate indication of the physical and chemical properties of the reaction medium over time would be very difficult and expensive, if not impossible. Thus, it is desirable to be able to accurately model the physical and chemical behavior of the reaction medium in a bubble column reactor without the need for acquiring extensive samples of the reaction medium.

### OBJECTS AND SUMMARY OF THE INVENTION

It is, therefore, an object of the present invention to provide a more effective and economical method for modeling liquid-phase oxidation in a bubble column reactor.

One embodiment of the present invention concerns a process comprising the following steps: (a) oxidizing an oxidizable compound in a liquid phase of an actual multi-phase reaction medium contained in an actual oxidation reactor; (b) determining at least one measured gas hold-up value for the actual reaction medium based on actual measurements taken during the oxidizing of step (a); (c) generating a computer model of a modeled oxidation reactor; (d) using the computer model to determine at least one modeled gas hold-up value for the modeled reaction medium; and (e) comparing the modeled and measured gas hold-up values to one another.

Another embodiment of the present invention concerns a process comprising the following steps: (a) oxidizing an oxidizable compound in a liquid phase of an actual multi-phase reaction medium contained in an actual oxidation reactor; (b) determining at least one measured reactant concentration value of the actual reaction medium based on actual measurements taken during the oxidizing of step (a); (c) generating a computer model of a modeled reaction medium contained in a modeled oxidation reactor; (d) using the computer model to determine at least one modeled reactant concentration value for the modeled reaction medium; and (e) comparing the modeled and measured reactant concentration values to one another.

Still another embodiment of the present invention concerns a process comprising the following steps: (a) oxidizing para-xylene in a liquid phase of an actual multi-phase reaction medium contained in an actual bubble column reactor; (b) determining at least one measured gas hold-up value and at least one measured reactant concentration value for the actual reaction medium based on actual measurements taken during the oxidizing of step (a); (c) generating a computer model of a modeled bubble column oxidation reactor containing a modeled multi-phase reaction medium; (d) using the computer model to determine at least one modeled gas hold-up value and at least one modeled reactant concentration value for the modeled reaction medium; and (e) adjusting one or more parameters of the computer model based on a comparison of the measured and modeled gas hold-up values and/or a comparison of the measure and modeled reactant concentration values.

### BRIEF DESCRIPTION OF THE DRAWINGS

Preferred embodiments of the invention are described in detail below with reference to the attached drawing figures, wherein;

FIG. 1 is a side view of an oxidation reactor which can be modeled in accordance with one embodiment of the present invention, particularly illustrating the introduction of feed, oxidant, and reflux streams into the reactor, the presence of a multi-phase reaction medium in the reactor, and the withdrawal of a gas and a slurry from the top and bottom of the reactor, respectively;

FIG. 2 is an enlarged sectional side view of the bottom of the bubble column reactor taken along line 2-2 in FIG. 3, particularly illustrating the location and configuration of an oxidant sparger used to introduce the oxidant stream into the reactor;

FIG. 3 is a top view of the oxidant sparger of FIG. 2, particularly illustrating the oxidant openings in the top of the oxidant sparger;

FIG. 4 is a bottom view of the oxidant sparger of FIG. 2, particularly illustrating the oxidant opening in the bottom of the oxidant sparger;

FIG. 5 is a sectional side view of the oxidant sparger taken along line 5-5 in FIG. 3, particularly illustrating the orientation of the oxidant openings in the top and bottom of the oxidant sparger;

FIG. 6 is an enlarged side view of the bottom portion of the bubble column reactor, particular illustrating a system for introducing the feed stream into the reactor at multiple, vertically-space locations;

FIG. 7 is a sectional top view taken along line 7-7 in FIG. 6, particularly illustrating how the feed introduction system shown in FIG. 6 distributes the feed stream into in a preferred radial feed zone (FZ) and more than one azimuthal quadrant (Q₁, Q₂, Q₃, Q₄);

FIG. 8 is a sectional top view similar to FIG. 7, but illustrating an alternative means for discharging the feed stream into the reactor using bayonet tubes each having a plurality of small feed openings;

FIG. 9 is an isometric view of an alternative system for introducing the feed stream into the reaction zone at multiple, vertically-space locations without requiring multiple vessel penetrations, particularly illustrating that the feed distribution system can be at least partly supported on the oxidant sparger;

FIG. 10 is a side view of the single-penetration feed distribution system and oxidant sparger illustrated in FIG. 9;

FIG. 11 is a sectional top view taken along line 11-11 in FIG. 10 and further illustrating the single-penetration feed distribution system supported on the oxidant sparger;

FIG. 12 is a side view of a bubble column reactor containing a multi-phase reaction medium, particularly illustrating the reaction medium being theoretically partitioned into 30 horizontal slices of equal volume in order to quantify certain gradients in the reaction medium;

FIG. 13 is a side view of a bubble column reactor containing a multi-phase reaction medium, particularly illustrating first and second discrete 20-percent continuous volumes of the reaction medium that have substantially different oxygen concentrations and/or oxygen consumption rates;

FIG. 14 is a side view of two stacked reaction vessels, with or without optional mechanical agitation, containing a multi-phase reaction medium, particularly illustrating that the vessels contain discrete 20-percent continuous volumes of the reaction medium having substantially different oxygen concentrations and/or oxygen consumption rates;

FIG. 15 is a side view of three side-by-side reaction vessels, with or without optional mechanical agitation, containing a multi-phase reaction medium, particularly illustrating that the vessels contain discrete 20-percent continuous volumes of the reaction medium having substantially different oxygen concentrations and/or oxygen consumption rates;

FIGS. 16A and 16B are magnified views of crude terephthalic acid (CTA) particles produced in accordance with one embodiment of the present invention, particularly illustrating that each CTA particle is a low density, high surface area particle composed of a plurality of loosely-bound CTA sub-particles;

FIGS. 17A and 17B are magnified views of a conventionally-produced CTA, particularly illustrating that the conventional CTA particle has a larger particle size, lower density, and lower surface area than the inventive CTA particle of FIGS. 16A and 16B;

FIG. 18 is a simplified process flow diagram of a prior art process for making purified terephthalic acid (PTA);

FIG. 19 is a simplified process flow diagram of a process for making PTA in accordance with one embodiment of the present invention;

FIG. 20a is the first part of a flow diagram outlining steps for modeling a bubble column oxidation reactor on a computer;

FIG. 20b is the second part of the flow diagram outlining steps for modeling the bubble column oxidation reactor on a computer; and

FIG. 20c is the third part of the flow diagram outlining steps for modeling the bubble column oxidation reactor on a computer.

### DETAILED DESCRIPTION

One embodiment of the present invention concerns a method for modeling the liquid-phase partial oxidation of an oxidizable compound. Such oxidation is preferably carried out in the liquid phase of a multi-phase reaction medium contained in one or more agitated reactors. Suitable agitated reactors include, for example, bubble-agitated reactors (e.g., bubble column reactors), mechanically agitated reactors (e.g., continuous stirred tank reactors), and flow agitated reactors (e.g., jet reactors). In one embodiment of the invention, the liquid-phase oxidation is carried out in a single bubble column reactor.

As used herein, the term “bubble column reactor” shall denote a reactor for facilitating chemical reactions in a multi-phase reaction medium, wherein agitation of the reaction medium is provided primarily by the upward movement of gas bubbles through the reaction medium. As used herein, the term “agitation” shall denote work dissipated into the reaction medium causing fluid flow and/or mixing. As used herein, the terms “majority,” “primarily,” and “predominately” shall mean more than 50 percent. As used herein, the term “mechanical agitation” shall denote agitation of the reaction medium caused by physical movement of a rigid or flexible element(s) against or within the reaction medium. For example, mechanical agitation can be provided by rotation, oscillation, and/or vibration of internal stirrers, paddles, vibrators, or acoustical diaphragms located in the reaction medium. As used herein, the term “flow agitation” shall denote agitation of the reaction medium caused by high velocity injection and/or recirculation of one or more fluids in the reaction medium. For example, flow agitation can be provided by nozzles, ejectors, and/or eductors.

In a preferred embodiment of the present invention, less than about 40 percent of the agitation of the reaction medium in the bubble column reactor during oxidation is provided by mechanical and/or flow agitation, more preferably less than about 20 percent of the agitation is provided by mechanical and/or flow agitation, and most preferably less than 5 percent of the agitation is provided by mechanical and/or flow agitation. Preferably, the amount of mechanical and/or flow agitation imparted to the multi-phase reaction medium during oxidation is less than about 3 kilowatts per cubic meter of the reaction medium, more preferably less than about 2 kilowatts per cubic meter, and most preferably less than 1 kilowatt per cubic meter.

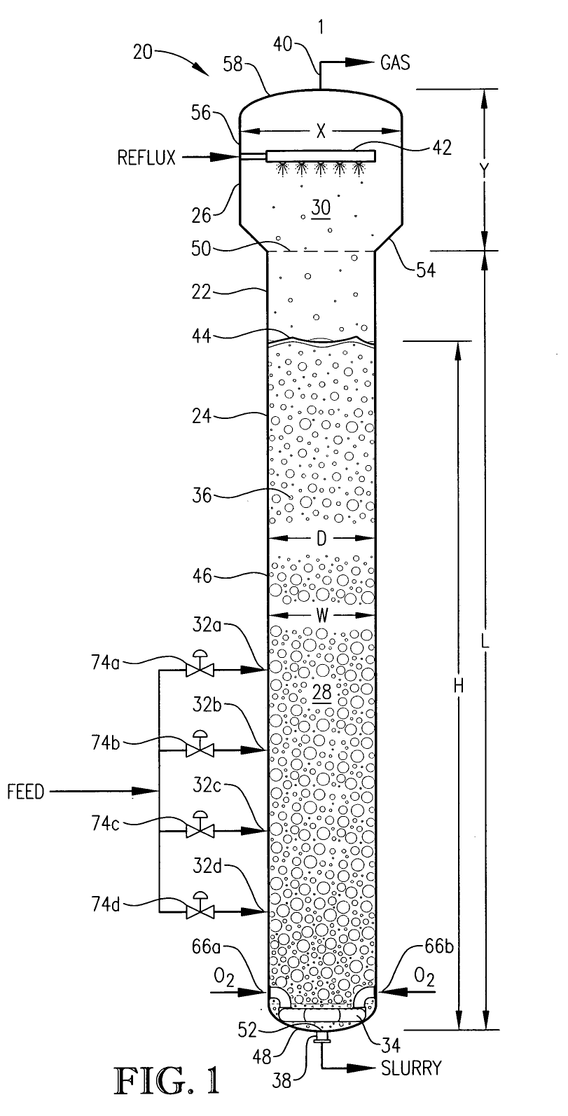

Referring now to FIG. 1, a preferred bubble column reactor 20 to be modeled is illustrated as comprising a vessel shell 22 having of a reaction section 24 and a disengagement section 26. Reaction section 24 defines an internal reaction zone 28, while disengagement section 26 defines an internal disengagement zone 30. A predominately liquid-phase feed stream is introduced into reaction zone 28 via feed inlets 32a,b,c,d. A predominately gas-phase oxidant stream is introduced into reaction zone 28 via an oxidant sparger 34 located in the lower portion of reaction zone 28. The liquid-phase feed stream and gas-phase oxidant stream cooperatively form a multi-phase reaction medium 36 within reaction zone 28. Multi-phase reaction medium 36 comprises a liquid phase and a gas phase. More preferably, multi-phase reaction medium 36 comprises a three-phase medium having solid-phase, liquid-phase, and gas-phase components. The solid-phase component of the reaction medium 36 preferably precipitates within reaction zone 28 as a result of the oxidation reaction carried out in the liquid phase of reaction medium 36. Bubble column reactor 20 includes a slurry outlet 38 located near the bottom of reaction zone 28 and a gas outlet 40 located near the top of disengagement zone 30. A slurry effluent comprising liquid-phase and solid-phase components of reaction medium 36 is withdrawn from reaction zone 28 via slurry outlet 38, while a predominantly gaseous effluent is withdrawn from disengagement zone 30 via gas outlet 40.

The liquid-phase feed stream introduced into bubble column reactor 20 via feed inlets 32a,b,c,d preferably comprises an oxidizable compound, a solvent, and a catalyst system.

The oxidizable compound present in the liquid-phase feed stream preferably comprises at least one hydrocarbyl group. More preferably, the oxidizable compound is an aromatic compound. Still more preferably, the oxidizable compound is an aromatic compound with at least one attached hydrocarbyl group or at least one attached substituted hydrocarbyl group or at least one attached heteroatom or at least one attached carboxylic acid function (—COOH). Even more preferably, the oxidizable compound is an aromatic compound with at least one attached hydrocarbyl group or at least one attached substituted hydrocarbyl group with each attached group comprising from 1 to 5 carbon atoms. Yet still more preferably, the oxidizable compound is an aromatic compound having exactly two attached groups with each attached group comprising exactly one carbon atom and consisting of methyl groups and/or substituted methyl groups and/or at most one carboxylic acid group. Even still more preferably, the oxidizable compound is para-xylene, meta-xylene, para-tolualdehyde, meta-tolualdehyde, para-toluic acid, meta-toluic acid, and/or acetaldehyde. Most preferably, the oxidizable compound is para-xylene.

A “hydrocarbyl group,” as defined herein, is at least one carbon atom that is bonded only to hydrogen atoms or to other carbon atoms. A “substituted hydrocarbyl group,” as defined herein, is at least one carbon atom bonded to at least one heteroatom and to at least one hydrogen atom. “Heteroatoms,” as defined herein, are all atoms other than carbon and hydrogen atoms. Aromatic compounds, as defined herein, comprise an aromatic ring, preferably having at least 6 carbon atoms, even more preferably having only carbon atoms as part of the ring. Suitable examples of such aromatic rings include, but are not limited to, benzene, biphenyl, terphenyl, naphthalene, and other carbon-based fused aromatic rings.

If the oxidizable compound present in the liquid-phase feed stream is a normally-solid compound (i.e., is a solid at standard temperature and pressure), it is preferred for the oxidizable compound to be substantially dissolved in the solvent when introduced into reaction zone 28. It is preferred for the boiling point of the oxidizable compound at atmospheric pressure to be at least about 50° C. More preferably, the boiling point of the oxidizable compound is in the range of from about 80 to about 400° C., and most preferably in the range of from 125 to 155° C. The amount of oxidizable compound present in the liquid-phase feed is preferably in the range of from about 2 to about 40 weight percent, more preferably in the range of from about 4 to about 20 weight percent, and most preferably in the range of from 6 to 15 weight percent.

It is now noted that the oxidizable compound present in the liquid-phase feed may comprise a combination of two or more different oxidizable chemicals. These two or more different chemical materials can be fed commingled in the liquid-phase feed stream or may be fed separately in multiple feed streams. For example, an oxidizable compound comprising para-xylene, meta-xylene, para-tolualdehyde, para-toluic acid, and acetaldehyde may be fed to the reactor via a single inlet or multiple separate inlets.

The solvent present in the liquid-phase feed stream preferably comprises an acid component and a water component. The solvent is preferably present in the liquid-phase feed stream at a concentration in the range of from about 60 to about 98 weight percent, more preferably in the range of from about 80 to about 96 weight percent, and most preferably in the range of from 85 to 94 weight percent. The acid component of the solvent is preferably primarily an organic low molecular weight monocarboxylic acid having 1-6 carbon atoms, more preferably 2 carbon atoms. Most preferably, the acid component of the solvent is primarily acetic acid. Preferably, the acid component makes up at least about 75 weight percent of the solvent, more preferably at least about 80 weight percent of the solvent, and most preferably 85 to 98 weight percent of the solvent, with the balance being primarily water. The solvent introduced into bubble column reactor 20 can include small quantities of impurities such as, for example, para-tolualdehyde, terephthaldehyde, 4-carboxybenzaldehyde (4-CBA), benzoic acid, para-toluic acid, para-toluic aldehyde, alpha-bromo-para-toluic acid, isophthalic acid, phthalic acid, trimellitic acid, polyaromatics, and/or suspended particulate. It is preferred that the total amount of impurities in the solvent introduced into bubble column reactor 20 is less than about 3 weight percent.

The catalyst system present in the liquid-phase feed stream is preferably a homogeneous, liquid-phase catalyst system capable of promoting oxidation (including partial oxidation) of the oxidizable compound. More preferably, the catalyst system comprises at least one multivalent transition metal. Still more preferably, the multivalent transition metal comprises cobalt. Even more preferably, the catalyst system comprises cobalt and bromine. Most preferably, the catalyst system comprises cobalt, bromine, and manganese.

When cobalt is present in the catalyst system, it is preferred for the amount of cobalt present in the liquid-phase feed stream to be such that the concentration of cobalt in the liquid phase of reaction medium 36 is maintained in the range of from about 300 to about 6,000 parts per million by weight (ppmw), more preferably in the range of from about 700 to about 4,200 ppmw, and most preferably in the range of from 1,200 to 3,000 ppmw. When bromine is present in the catalyst system, it is preferred for the amount of bromine present in the liquid-phase feed stream to be such that the concentration of bromine in the liquid phase of reaction medium 36 is maintained in the range of from about 300 to about 5,000 ppmw, more preferably in the range of from about 600 to about 4,000 ppmw, and most preferably in the range of from 900 to 3,000 ppmw. When manganese is present in the catalyst system, it is preferred for the amount of manganese present in the liquid-phase feed stream to be such that the concentration of manganese in the liquid phase of reaction medium 36 is maintained in the range of from about 20 to about 1,000 ppmw, more preferably in the range of from about 40 to about 500 ppmw, most preferably in the range of from 50 to 200 ppmw.

The concentrations of the cobalt, bromine, and/or manganese in the liquid phase of reaction medium 36, provided above, are expressed on a time-averaged and volume-averaged basis. As used herein, the term “time-averaged” shall denote an average of at least 10 measurements taken equally over a continuous period of at least 100 seconds. As used herein, the term “volume-averaged” shall denote an average of at least 10 measurements taken at uniform 3-dimensional spacing throughout a certain volume.

The weight ratio of cobalt to bromine (Co:Br) in the catalyst system introduced into reaction zone 28 is preferably in the range of from about 0.25:1 to about 4:1, more preferably in the range of from about 0.5:1 to about 3:1, and most preferably in the range of from 0.75:1 to 2:1. The weight ratio of cobalt to manganese (Co:Mn) in the catalyst system introduced into reaction zone 28 is preferably in the range of from about 0.3:1 to about 40:1, more preferably in the range of from about 5:1 to about 30:1, and most preferably in the range of from 10:1 to 25:1.

The liquid-phase feed stream introduced into bubble column reactor 20 can include small quantities of impurities such as, for example, toluene, ethylbenzene, para-tolualdehyde, terephthaldehyde, 4-carboxybenzaldehyde (4-CBA), benzoic acid, para-toluic acid, para-toluic aldehyde, alpha bromo para-toluic acid, isophthalic acid, phthalic acid, trimellitic acid, polyaromatics, and/or suspended particulate. When bubble column reactor 20 is employed for the production of terephthalic acid, meta-xylene and ortho-xylene are also considered impurities. It is preferred that the total amount of impurities in the liquid-phase feed stream introduced into bubble column reactor 20 is less than about 3 weight percent.

Although FIG. 1 illustrates an embodiment where the oxidizable compound, the solvent, and the catalyst system are mixed together and introduced into bubble column reactor 20 as a single feed stream, in an alternative embodiment of the present invention, the oxidizable compound, the solvent, and the catalyst can be separately introduced into bubble column reactor 20. For example, it is possible to feed a pure para-xylene stream into bubble column reactor 20 via an inlet separate from the solvent and catalyst inlet(s).

The predominately gas-phase oxidant stream introduced into bubble column reactor 20 via oxidant sparger 34 comprises molecular oxygen (O₂). Preferably, the oxidant stream comprises in the range of from about 5 to about 40 mole percent molecular oxygen, more preferably in the range of from about 15 to about 30 mole percent molecular oxygen, and most preferably in the range of from 18 to 24 mole percent molecular oxygen. It is preferred for the balance of the oxidant stream to be comprised primarily of a gas or gasses, such as nitrogen, that are inert to oxidation. More preferably, the oxidant stream consists essentially of molecular oxygen and nitrogen. Most preferably, the oxidant stream is dry air that comprises about 21 mole percent molecular oxygen and about 78 to about 81 mole percent nitrogen. In an alternative embodiment of the present invention, the oxidant stream can comprise substantially pure oxygen.

Referring again to FIG. 1, bubble column reactor 20 is preferably equipped with a reflux distributor 42 positioned above an upper surface 44 of reaction medium 36. Reflux distributor 42 is operable to introduce droplets of a predominately liquid-phase reflux stream into disengagement zone 30 by any means of droplet formation known in the art. More preferably, reflux distributor 42 produces a spray of droplets directed downwardly towards upper surface 44 of reaction medium 36. Preferably, this downward spray of droplets affects (i.e., engages and influences) at least about 50 percent of the maximum horizontal cross-sectional area of disengagement zone 30. More preferably, the spray of droplets affects at least about 75 percent of the maximum horizontal cross-sectional area of disengagement zone 30. Most preferably, the spray of droplets affects at least 90 percent of the maximum horizontal cross-sectional area of disengagement zone 30. This downward liquid reflux spray can help prevent foaming at or above upper surface 44 of reaction medium 36 and can also aid in the disengagement of any liquid or slurry droplets entrained in the upwardly moving gas that flows towards gas outlet 40. Further, the liquid reflux may serve to reduce the amount of particulates and potentially precipitating compounds (e.g., dissolved benzoic acid, para-toluic acid, 4-CBA, terephthalic acid, and catalyst metal salts) exiting in the gaseous effluent withdrawn from disengagement zone 30 via gas outlet 40. In addition, the introduction of reflux droplets into disengagement zone 30 can, by a distillation action, be used to adjust the composition of the gaseous effluent withdrawn via gas outlet 40.

The liquid reflux stream introduced into bubble column reactor 20 via reflux distributor 42 preferably has about the same composition as the solvent component of the liquid-phase feed stream introduced into bubble column reactor 20 via feed inlets 32a,b,c,d. Thus, it is preferred for the liquid reflux stream to comprise an acid component and water. The acid component of the reflux stream is preferably a low molecular weight organic monocarboxylic acid having 1-6 carbon atoms, more preferably 2 carbon atoms. Most preferably, the acid component of the reflux stream is acetic acid. Preferably, the acid component makes up at least about 75 weight percent of the reflux stream, more preferably at least about 80 weight percent of the reflux stream, and most preferably 85 to 98 weight percent of the reflux stream, with the balance being water. Because the reflux stream typically has substantially the same composition as the solvent in the liquid-phase feed stream, when this description refers to the “total solvent” introduced into the reactor, such “total solvent” shall include both the reflux stream and the solvent portion of the feed stream.

During liquid-phase oxidation in bubble column reactor 20, it is preferred for the feed, oxidant, and reflux streams to be substantially continuously introduced into reaction zone 28, while the gas and slurry effluent streams are substantially continuously withdrawn from reaction zone 28. As used herein, the term “substantially continuously” shall mean for a period of at least 10 hours interrupted by less than 10 minutes. During oxidation, it is preferred for the oxidizable compound (e.g., para-xylene) to be substantially continuously introduced into reaction zone 28 at a rate of at least about 8,000 kilograms per hour, more preferably at a rate in the range of from about 13,000 to about 80,000 kilograms per hour, still more preferably in the range of from about 18,000 to about 50,000 kilograms per hour, and most preferably in the range of from 22,000 to 30,000 kilograms per hour. Although it is generally preferred for the flow rates of the incoming feed, oxidant, and reflux streams to be substantially steady, it is now noted that one embodiment of the presenting invention contemplates pulsing the incoming feed, oxidant, and/or reflux stream in order to improve mixing and mass transfer. When the incoming feed, oxidant, and/or reflux stream are introduced in a pulsed fashion, it is preferred for their flow rates to vary within about 0 to about 500 percent of the steady-state flow rates recited herein, more preferably within about 30 to about 200 percent of the steady-state flow rates recited herein, and most preferably within 80 to 120 percent of the steady-state flow rates recited herein.

The average space-time rate of reaction (STR) in bubble column oxidation reactor 20 is defined as the mass of the oxidizable compound fed per unit volume of reaction medium 36 per unit time (e.g., kilograms of para-xylene fed per cubic meter per hour). In conventional usage, the amount of oxidizable compound not converted to product would typically be subtracted from the amount of oxidizable compound in the feed stream before calculating the STR. However, conversions and yields are typically high for many of the oxidizable compounds preferred herein (e.g., para-xylene), and it is convenient to define the term herein as stated above. For reasons of capital cost and operating inventory, among others, it is generally preferred that the reaction be conducted with a high STR. However, conducting the reaction at increasingly higher STR may affect the quality or yield of the partial oxidation. Bubble column reactor 20 is particularly useful when the STR of the oxidizable compound (e.g., para-xylene) is in the range of from about 25 kilograms per cubic meter per hour to about 400 kilograms per cubic meter per hour, more preferably in the range of from about 30 kilograms per cubic meter per hour to about 250 kilograms per cubic meter per hour, still more preferably from about 35 kilograms per cubic meter per hour to about 150 kilograms per cubic meter per hour, and most preferably in the range of from 40 kilograms per cubic meter per hour to 100 kilograms per cubic meter per hour.

The oxygen-STR in bubble column oxidation reactor 20 is defined as the weight of molecular oxygen consumed per unit volume of reaction medium 36 per unit time (e.g., kilograms of molecular oxygen consumed per cubic meter per hour). For reasons of capital cost and oxidative consumption of solvent, among others, it is generally preferred that the reaction be conducted with a high oxygen-STR. However, conducting the reaction at increasingly higher oxygen-STR eventually reduces the quality or yield of the partial oxidation. Without being bound by theory, it appears that this possibly relates to the transfer rate of molecular oxygen from the gas phase into the liquid at the interfacial surface area and thence into the bulk liquid. Too high an oxygen-STR possibly leads to too low a dissolved oxygen content in the bulk liquid phase of the reaction medium.

The global-average-oxygen-STR is defined herein as the weight of all oxygen consumed in the entire volume of reaction medium 36 per unit time (e.g., kilograms of molecular oxygen consumed per cubic meter per hour). Bubble column reactor 20 is particularly useful when the global-average-oxygen-STR is in the range of from about 25 kilograms per cubic meter per hour to about 400 kilograms per cubic meter per hour, more preferably in the range of from about 30 kilograms per cubic meter per hour to about 250 kilograms per cubic meter per hour, still more preferably from about 35 kilograms per cubic meter per hour to about 150 kilograms per cubic meter per hour, and most preferably in the range of from 40 kilograms per cubic meter per hour to 100 kilograms per cubic meter per hour.

During oxidation in bubble column reactor 20, it is preferred for the ratio of the mass flow rate of the total solvent (from both the feed and reflux streams) to the mass flow rate of the oxidizable compound entering reaction zone 28 to be maintained in the range of from about 2:1 to about 50:1, more preferably in the range of from about 5:1 to about 40:1, and most preferably in the range of from 7.5:1 to 25:1. Preferably, the ratio of the mass flow rate of solvent introduced as part of the feed stream to the mass flow rate of solvent introduced as part of the reflux stream is maintained in the range of from about 0.5:1 to no reflux stream flow whatsoever, more preferably in the range of from about 0.5:1 to about 4:1, still more preferably in the range of from about 1:1 to about 2:1, and most preferably in the range of from 1.25:1 to 1.5:1.

During liquid-phase oxidation in bubble column reactor 20, it is preferred for the oxidant stream to be introduced into bubble column reactor 20 in an amount that provides molecular oxygen somewhat exceeding the stoichiometric oxygen demand. The amount of excess molecular oxygen required for best results with a particular oxidizable compound affects the overall economics of the liquid-phase oxidation. During liquid-phase oxidation in bubble column reactor 20, it is preferred that the ratio of the mass flow rate of the oxidant stream to the mass flow rate of the oxidizable organic compound (e.g., para-xylene) entering reactor 20 is maintained in the range of from about 0.5:1 to about 20:1, more preferably in the range of from about 1:1 to about 10:1, and most preferably in the range of from 2:1 to 6:1.

Referring again to FIG. 1, the feed, oxidant, and reflux streams introduced into bubble column reactor 20 cooperatively form at least a portion of multi-phase reaction medium 36. Reaction medium 36 is preferably a three-phase medium comprising a solid phase, a liquid phase, and a gas phase. As mentioned above, oxidation of the oxidizable compound (e.g., para-xylene) takes place predominately in the liquid phase of reaction medium 36. Thus, the liquid phase of reaction medium 36 comprises dissolved oxygen and the oxidizable compound. The exothermic nature of the oxidation reaction that takes place in bubble column reactor 20 causes a portion of the solvent (e.g., acetic acid and water) introduced via feed inlets 32a,b,c,d to boil/vaporize. Thus, the gas phase of reaction medium 36 in reactor 20 is formed primarily of vaporized solvent and an undissolved, unreacted portion of the oxidant stream. Certain prior art oxidation reactors employ heat exchange tubes/fins to heat or cool the reaction medium. However, such heat exchange structures may be undesirable in the inventive reactor and process described herein. Thus, it is preferred for bubble column reactor 20 to include substantially no surfaces that contact reaction medium 36 and exhibit a time-averaged heat flux greater than 30,000 watts per meter squared.

The concentration of dissolved oxygen in the liquid phase of reaction medium 36 is a dynamic balance between the rate of mass transfer from the gas phase and the rate of reactive consumption within the liquid phase (i.e. it is not set simply by the partial pressure of molecular oxygen in the supplying gas phase, though this is one factor in the supply rate of dissolved oxygen and it does affect the limiting upper concentration of dissolved oxygen). The amount of dissolved oxygen varies locally, being higher near bubble interfaces. Globally, the amount of dissolved oxygen depends on the balance of supply and demand factors in different regions of reaction medium 36. Temporally, the amount of dissolved oxygen depends on the uniformity of gas and liquid mixing relative to chemical consumption rates. In designing to match appropriately the supply of and demand for dissolved oxygen in the liquid phase of reaction medium 36, it is preferred for the time-averaged and volume-averaged oxygen concentration in the liquid phase of reaction medium 36 to be maintained above about 1 ppm molar, more preferably in the range from about 4 to about 1,000 ppm molar, still more preferably in the range from about 8 to about 500 ppm molar, and most preferably in the range from 12 to 120 ppm molar.

The liquid-phase oxidation reaction carried out in bubble column reactor 20 is preferably a precipitating reaction that generates solids. More preferably, the liquid-phase oxidation carried out in bubble column reactor 20 causes at least about 10 weight percent of the oxidizable compound (e.g., para-xylene) introduced into reaction zone 28 to form a solid compound (e.g., crude terephthalic acid particles) in reaction medium 36. Still more preferably, the liquid-phase oxidation causes at least about 50 weight percent of the oxidizable compound to form a solid compound in reaction medium 36. Most preferably, the liquid-phase oxidation causes at least 90 weight percent of the oxidizable compound to form a solid compound in reaction medium 36. It is preferred for the total amount of solids in reaction medium 36 to be greater than about 3 percent by weight on a time-averaged and volume-averaged basis. More preferably, the total amount of solids in reaction medium 36 is maintained in the range of from about 5 to about 40 weight percent, still more preferably in the range of from about 10 to about 35 weight percent, and most preferably in the range of from 15 to 30 weight percent. It is preferred for a substantial portion of the oxidation product (e.g., terephthalic acid) produced in bubble column reactor 20 to be present in reaction medium 36 as solids, as opposed to remaining dissolved in the liquid phase of reaction medium 36. The amount of the solid phase oxidation product present in reaction medium 36 is preferably at least about 25 percent by weight of the total oxidation product (solid and liquid phase) in reaction medium 36, more preferably at least about 75 percent by weight of the total oxidation product in reaction medium 36, and most preferably at least 95 percent by weight of the total oxidation product in reaction medium 36. The numerical ranges provided above for the amount of solids in reaction medium 36 apply to substantially steady-state operation of bubble column 20 over a substantially continuous period of time, not to start-up, shut-down, or sub-optimal operation of bubble column reactor 20. The amount of solids in reaction medium 36 is determined by a gravimetric method. In this gravimetric method, a representative portion of slurry is withdrawn from the reaction medium and weighed. At conditions that effectively maintain the overall solid-liquid partitioning present within the reaction medium, free liquid is removed from the solids portion by sedimentation or filtration, effectively without loss of precipitated solids and with less than about 10 percent of the initial liquid mass remaining with the portion of solids. The remaining liquid on the solids is evaporated to dryness, effectively without sublimation of solids. The remaining portion of solids is weighed. The ratio of the weight of the portion of solids to the weight of the original portion of slurry is the fraction of solids, typically expressed as a percentage.

The precipitating reaction carried out in bubble column reactor 20 can cause fouling (i.e., solids build-up) on the surface of certain rigid structures that contact reaction medium 36. Thus, in one embodiment of the present invention, it is preferred for bubble column reactor 20 to include substantially no internal heat exchange, stirring, or baffling structures in reaction zone 28 because such structures would be prone to fouling. If internal structures are present in reaction zone 28, it is desirable to avoid internal structures having outer surfaces that include a significant amount of upwardly facing planar surface area because such upwardly facing planar surfaces would be highly prone to fouling. Thus, if any internal structures are present in reaction zone 28, it is preferred for less than about 20 percent of the total upwardly facing exposed outer surface area of such internal structures to be formed by substantially planar surfaces inclined less than about 15 degrees from horizontal.

Referring again to FIG. 1, the physical configuration of bubble column reactor 20 helps provide for optimized oxidation of the oxidizable compound (e.g., para-xylene) with minimal impurity generation. It is preferred for elongated reaction section 24 of vessel shell 22 to include a substantially cylindrical main body 46 and a lower head 48. The upper end of reaction zone 28 is defined by a horizontal plane 50 extending across the top of cylindrical main body 46. A lower end 52 of reaction zone 28 is defined by the lowest internal surface of lower head 48. Typically, lower end 52 of reaction zone 28 is located proximate the opening for slurry outlet 38. Thus, elongated reaction zone 28 defined within bubble column reactor 20 has a maximum length “L” measured from the top end 50 to the bottom end 52 of reaction zone 28 along the axis of elongation of cylindrical main body 46. The length “L” of reaction zone 28 is preferably in the range of from about 10 to about 100 meters, more preferably in the range of from about 20 to about 75 meters, and most preferably in the range of from 25 to 50 meters. Reaction zone 28 has a maximum diameter (width) “D” that is typically equal to the maximum internal diameter of cylindrical main body 46. The maximum diameter “D” of reaction zone 28 is preferably in the range of from about 1 to about 12 meters, more preferably in the range of from about 2 to about 10 meters, still more preferably in the range of from about 3.1 to about 9 meters, and most preferably in the range of from 4 to 8 meters. In a preferred embodiment of the present invention, reaction zone 28 has a length-to-diameter “L:D” ratio in the range of from about 6:1 to about 30:1. Still more preferably, reaction zone 28 has an L:D ratio in the range of from about 8:1 to about 20:1. Most preferably, reaction zone 28 has an L:D ratio in the range of from 9:1 to 15:1.

As discussed above, reaction zone 28 of bubble column reactor 20 receives multi-phase reaction medium 36. Reaction medium 36 has a bottom end coincident with lower end 52 of reaction zone 28 and a top end located at upper surface 44. Upper surface 44 of reaction medium 36 is defined along a horizontal plane that cuts through reaction zone 28 at a vertical location where the contents of reaction zone 28 transitions from a gas-phase-continuous state to a liquid-phase-continuous state. Upper surface 44 is preferably positioned at the vertical location where the local time-averaged gas hold-up of a thin horizontal slice of the contents of reaction zone 28 is 0.9.

Reaction medium 36 has a maximum height “H” measured between its upper and lower ends. The maximum width “W” of reaction medium 36 is typically equal to the maximum diameter “D” of cylindrical main body 46. During liquid-phase oxidation in bubble column reactor 20, it is preferred that H is maintained at about 60 to about 120 percent of L, more preferably about 80 to about 110 percent of L, and most preferably 85 to 100 percent of L. In a preferred embodiment of the present invention, reaction medium 36 has a height-to-width “H:W” ratio greater than about 3:1. More preferably, reaction medium 36 has an H:W ratio in the range of from about 7:1 to about 25:1. Still more preferably, reaction medium 36 has an H:W ratio in the range of from about 8:1 to about 20:1. Most preferably, reaction medium 36 has an H:W ratio in the range of from 9:1 to 15:1. In one embodiment of the invention, L=H and D=W so that various dimensions or ratios provide herein for L and D also apply to H and W, and vice-versa.

The relatively high L:D and H:W ratios provided in accordance with an embodiment of the invention can contribute to several important advantages of the inventive system. As discussed in further detail below, it has been discovered that higher L:D and H:W ratios, as well as certain other features discussed below, can promote beneficial vertical gradients in the concentrations of molecular oxygen and/or the oxidizable compound (e.g., para-xylene) in reaction medium 36. Contrary to conventional wisdom, which would favor a well-mixed reaction medium with relatively uniform concentrations throughout, it has been discovered that the vertical staging of the oxygen and/or the oxidizable compound concentrations facilitates a more effective and economical oxidation reaction. Minimizing the oxygen and oxidizable compound concentrations near the top of reaction medium 36 can help avoid loss of unreacted oxygen and unreacted oxidizable compound through upper gas outlet 40. However, if the concentrations of oxidizable compound and unreacted oxygen are low throughout reaction medium 36, then the rate and/or selectivity of oxidation are reduced. Thus, it is preferred for the concentrations of molecular oxygen and/or the oxidizable compound to be significantly higher near the bottom of reaction medium 36 than near the top of reaction medium 36.

In addition, high L:D and H:W ratios cause the pressure at the bottom of reaction medium 36 to be substantially greater than the pressure at the top of reaction medium 36. This vertical pressure gradient is a result of the height and density of reaction medium 36. One advantage of this vertical pressure gradient is that the elevated pressure at the bottom of the vessel drives more oxygen solubility and mass transfer than would otherwise be achievable at comparable temperatures and overhead pressures in shallow reactors. Thus, the oxidation reaction can be carried out at lower temperatures than would be required in a shallower vessel. When bubble column reactor 20 is used for the partial oxidation of para-xylene to crude terephthalic acid (CTA), the ability to operate at lower reaction temperatures with the same or better oxygen mass transfer rates has a number of advantages. For example, low temperature oxidation of para-xylene reduces the amount of solvent burned during the reaction. As discussed in further detail below, low temperature oxidation also favors the formation of small, high surface area, loosely bound, easily dissolved CTA particles, which can be subjected to more economical purification techniques than the large, low surface area, dense CTA particles produced by conventional high temperature oxidation processes.

During oxidation in reactor 20, it is preferred for the time-averaged and volume-averaged temperature of reaction medium 36 to be maintained in the range of from about 125 to about 200° C., more preferably in the range of from about 140 to about 180° C., and most preferably in the range of from 150 to 170° C. The overhead pressure above reaction medium 36 is preferably maintained in the range of from about 1 to about 20 bar gauge (barg), more preferably in the range of from about 2 to about 12 barg, and most preferably in the range of from 4 to 8 barg. Preferably, the pressure difference between the top of reaction medium 36 and the bottom of reaction medium 36 is in the range of from about 0.4 to about 5 bar, more preferably the pressure difference is in the range of from about 0.7 to about 3 bars, and most preferably the pressure difference is 1 to 2 bar. Although it is generally preferred for the overhead pressure above reaction medium 36 to be maintained at a relatively constant value, one embodiment of the present invention contemplates pulsing the overhead pressure to facilitate improved mixing and/or mass transfer in reaction medium 36. When the overhead pressure is pulsed, it is preferred for the pulsed pressures to range between about 60 to about 140 percent of the steady-state overhead pressure recited herein, more preferably between about 85 and about 115 percent of the steady-state overhead pressure recited herein, and most preferably between 95 and 105 percent of the steady-state overhead pressure recited herein.

A further advantage of the high L:D ratio of reaction zone 28 is that it can contribute to an increase in the average superficial velocity of reaction medium 36. The term “superficial velocity” and “superficial gas velocity,” as used herein with reference to reaction medium 36, shall denote the volumetric flow rate of the gas phase of reaction medium 36 at an elevation in the reactor divided by the horizontal cross-sectional area of the reactor at that elevation. The increased superficial velocity provided by the high L:D ratio of reaction zone 28 can promote local mixing and increase the gas hold-up of reaction medium 36. The time-averaged superficial velocities of reaction medium 36 at one-quarter height, half height, and/or three-quarter height of reaction medium 36 are preferably greater than about 0.3 meters per second, more preferably in the range of from about 0.8 to about 5 meters per second, still more preferably in the range of from about 0.9 to about 4 meters per second, and most preferably in the range of from 1 to 3 meters per second.

Referring again to FIG. 1, disengagement section 26 of bubble column reactor 20 is simply a widened portion of vessel shell 22 located immediately above reaction section 24. Disengagement section 26 reduces the velocity of the upwardly-flowing gas phase in bubble column reactor 20 as the gas phase rises above the upper surface 44 of reaction medium 36 and approaches gas outlet 40. This reduction in the upward velocity of the gas phase helps facilitate removal of entrained liquids and/or solids in the upwardly flowing gas phase and thereby reduces undesirable loss of certain components present in the liquid phase of reaction medium 36.

Disengagement section 26 preferably includes a generally frustoconical transition wall 54, a generally cylindrical broad sidewall 56, and an upper head 58. The narrow lower end of transition wall 54 is coupled to the top of cylindrical main body 46 of reaction section 24. The wide upper end of transition wall 54 is coupled to the bottom of broad sidewall 56. It is preferred for transition wall 54 to extend upwardly and outwardly from its narrow lower end at an angle in the range of from about 10 to about 70 degrees from vertical, more preferably in the range of about 15 to about 50 degrees from vertical, and most preferably in the range of from 15 to 45 degrees from vertical. Broad sidewall 56 has a maximum diameter “X” that is generally greater than the maximum diameter “D” of reaction section 24, though when the upper portion of reaction section 24 has a smaller diameter than the overall maximum diameter of reaction section 24, then X may actually be smaller than D. In a preferred embodiment of the present invention, the ratio of the diameter of broad sidewall 56 to the maximum diameter of reaction section 24 “X:D” is in the range of from about 0.8:1 to about 4:1, most preferably in the range of from 1.1:1 to 2:1. Upper head 58 is coupled to the top of broad sidewall 56. Upper head 58 is preferably a generally elliptical head member defining a central opening that permits gas to escape disengagement zone 30 via gas outlet 40. Alternatively, upper head 58 may be of any shape, including conical. Disengagement zone 30 has a maximum height “Y” measured from the top 50 of reaction zone 28 to the upper most portion of disengagement zone 30. The ratio of the length of reaction zone 28 to the height of disengagement zone 30 “L:Y” is preferably in the range of from about 2:1 to about 24:1, more preferably in the range of from about 3:1 to about 20:1, and most preferably in the range of from 4:1 to 16:1.

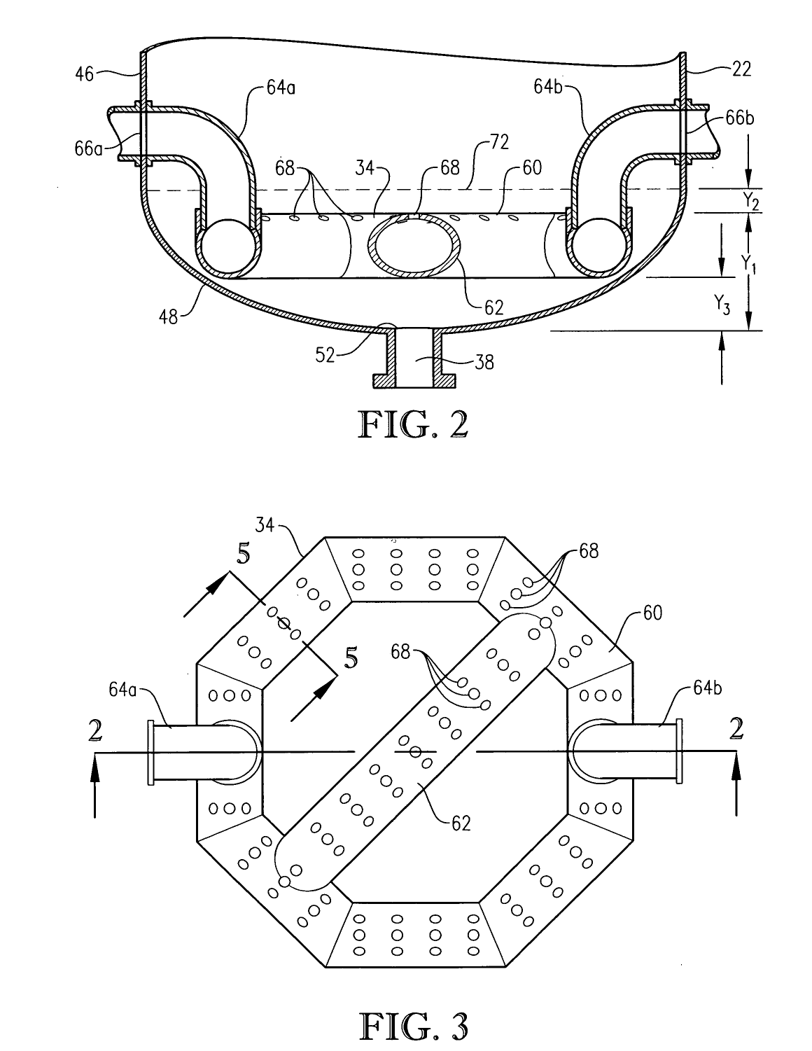

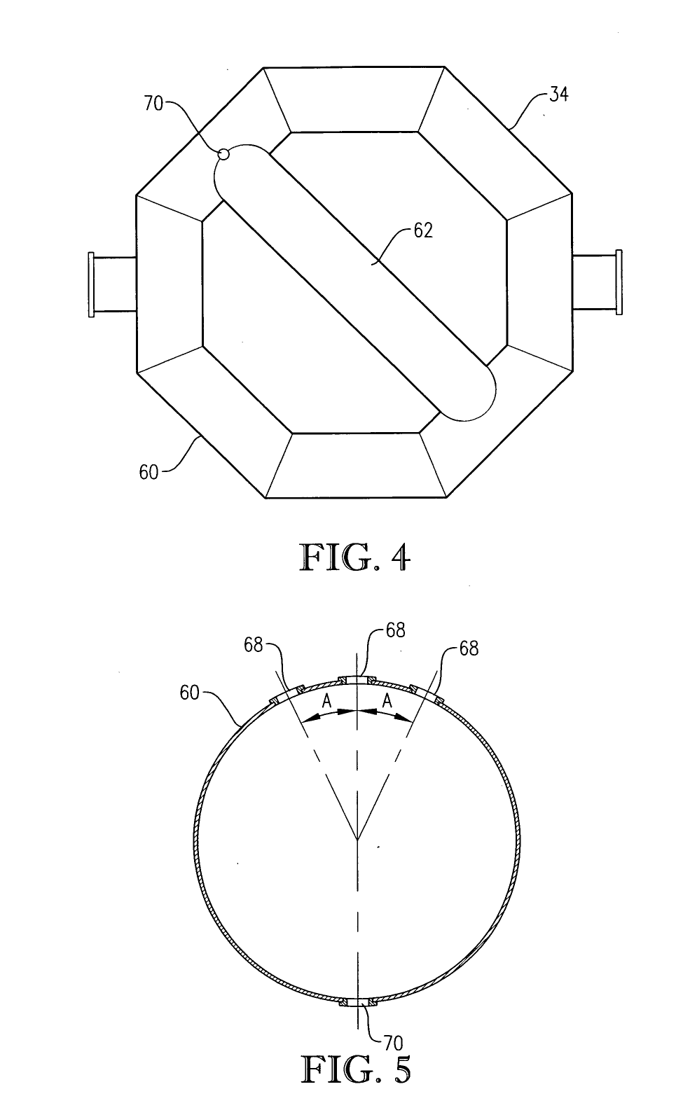

Referring now to FIGS. 1-5, the location and configuration of oxidant sparger 34 will now be discussed in greater detail. FIGS. 2 and 3 show that oxidant sparger 34 can include a ring member 60, a cross-member 62, and a pair of oxidant entry conduits 64a,b. Conveniently, these oxidant entry conduits 64a,b can enter the vessel at an elevation above the ring member 60 and then turn downwards as shown in FIGS. 2 and 3. Alternatively, an oxidant entry conduit 64a,b may enter the vessel below the ring member 60 or on about the same horizontal plane as ring member 60. Each oxidant entry conduit 64a,b includes a first end coupled to a respective oxidant inlet 66a,b formed in the vessel shell 22 and a second end fluidly coupled to ring member 60. Ring member 60 is preferably formed of conduits, more preferably of a plurality of straight conduit sections, and most preferably a plurality of straight pipe sections, rigidly coupled to one another to form a tubular polygonal ring. Preferably, ring member 60 is formed of at least 3 straight pipe sections, more preferably 6 to 10 pipe sections, and most preferably 8 pipe sections. Accordingly, when ring member 60 is formed of 8 pipe sections, it has a generally octagonal configuration. Cross-member 62 is preferably formed of a substantially straight pipe section that is fluidly coupled to and extends diagonally between opposite pipe sections of ring member 60. The pipe section used for cross-member 62 preferably has substantially the same diameter as the pipe sections used to form ring member 60. It is preferred for the pipe sections that make up oxidant entry conduits 64a,b, ring member 60, and cross-member 62 to have a nominal diameter greater than about 0.1 meter, more preferable in the range of from about 0.2 to about 2 meters, and most preferably in the range of from 0.25 to 1 meters. As perhaps best illustrated in FIG. 3, ring member 60 and cross-member 62 each present a plurality of upper oxidant openings 68 for discharging the oxidant stream upwardly into reaction zone 28. As perhaps best illustrated in FIG. 4, ring member 60 and/or cross-member 62 can present one or more lower oxidant openings 70 for discharging the oxidant stream downwardly into reaction zone 28. Lower oxidant openings 70 can also be used to discharge liquids and/or solids that might intrude within ring member 60 and/or cross-member 62. In order to prevent solids from building up inside oxidant sparger 34, a liquid stream can be continuously or periodically passed through sparger 34 to flush out any accumulated solids.

Referring again to FIGS. 1-4, during oxidation in bubble column reactor 20, oxidant streams are forced through oxidant inlets 66a,b and into oxidant entry conduits 64a,b, respectively. The oxidant streams are then transported via oxidant entry conduits 64a,b to ring member 60. Once the oxidant stream has entered ring member 60, the oxidant stream is distributed throughout the internal volumes of ring member 60 and cross-member 62. The oxidant stream is then forced out of oxidant sparger 34 and into reaction zone 28 via upper and lower oxidant openings 68,70 of ring member 60 and cross-member 62.

The outlets of upper oxidant openings 68 are laterally spaced from one another and are positioned at substantially the same elevation in reaction zone 28. Thus, the outlets of upper oxidant openings 68 are generally located along a substantially horizontal plane defined by the top of oxidant sparger 34. The outlets of lower oxidant openings 70 are laterally spaced from one another and are positioned at substantially the same elevation in reaction zone 28. Thus, the outlets of lower oxidant openings 70 are generally located along a substantially horizontal plane defined by the bottom of oxidant sparger 34.

In one embodiment of the present invention, oxidant sparger 34 has at least about 20 upper oxidant openings 68 formed therein. More preferably, oxidant sparger 34 has in the range of from about 40 to about 800 upper oxidant openings 68 formed therein. Most preferably, oxidant sparger 34 has in the range of from 60 to 400 upper oxidant openings 68 formed therein. Oxidant sparger 34 preferably has at least about 1 lower oxidant opening 70 formed therein. More preferably, oxidant sparger 34 has in the range of from about 2 to about 40 lower oxidant openings 70 formed therein. Most preferably, oxidant sparger 34 has in the range of from 8 to 20 lower oxidant openings 70 formed therein. The ratio of the number of upper oxidant openings 68 to lower oxidant openings 70 in oxidant sparger 34 is preferably in the range of from about 2:1 to about 100:1, more preferably in the range of from about 5:1 to about 25:1, and most preferably in the range of from 8:1 to 15:1. The diameters of substantially all upper and lower oxidant openings 68,70 are preferably substantially the same, so that the ratio of the volumetric flow rate of the oxidant stream out of upper and lower openings 68,70 is substantially the same as the ratios, given above, for the relative number of upper and lower oxidant openings 68,70.

FIG. 5 illustrates the direction of oxidant discharge from upper and lower oxidant openings 68,70. With reference to upper oxidant openings 68, it is preferred for at least a portion of upper oxidant openings 68 to discharge the oxidant stream in at an angle “A” that is skewed from vertical. It is preferred for the percentage of upper oxidant openings 68 that are skewed from vertical by angle “A” to be in the range of from about 30 to about 90 percent, more preferably in the range of from about 50 to about 80 percent, still more preferably in the range of from 60 to 75 percent, and most preferably about 67 percent. The angle “A” is preferably in the range of from about 5 to about 60 degrees, more preferably in the range of from about 10 to about 45 degrees, and most preferably in the range of from 15 to 30 degrees. As for lower oxidant openings 70, it is preferred that substantially all of lower oxidant openings 70 are located near the bottom-most portion of the ring member 60 and/or cross-member 62. Thus, any liquids and/or solids that may have unintentionally entered oxidant sparger 34 can be readily discharged from oxidant sparger 34 via lower oxidant openings 70. Preferably, lower oxidant openings 70 discharge the oxidant stream downwardly at a substantially vertical angle. For purposes of this description, an upper oxidant opening can be any opening that discharges an oxidant stream in a generally upward direction (i.e., at an angle above horizontal), and a lower oxidant opening can be any opening that discharges an oxidant stream in a generally downward direction (i.e., at an angle below horizontal).

In many conventional bubble column reactors containing a multi-phase reaction medium, substantially all of the reaction medium located below the oxidant sparger (or other mechanism for introducing the oxidant stream into the reaction zone) has a very low gas hold-up value. As known in the art, “gas hold-up” is simply the volume fraction of a multi-phase medium that is in the gaseous state. Zones of low gas hold-up in a medium can also be referred to as “unaerated” zones. In many conventional slurry bubble column reactors, a significant portion of the total volume of the reaction medium is located below the oxidant sparger (or other mechanism for introducing the oxidant stream into the reaction zone). Thus, a significant portion of the reaction medium present at the bottom of conventional bubble column reactors is unaerated.

It has been discovered that minimizing the amount of unaerated zones in a reaction medium subjected to oxidization in a bubble column reactor can minimize the generation of certain types of undesirable impurities. Unaerated zones of a reaction medium contain relatively few oxidant bubbles. This low volume of oxidant bubbles reduces the amount of molecular oxygen available for dissolution into the liquid phase of the reaction medium. Thus, the liquid phase in an unaerated zone of the reaction medium has a relatively low concentration of molecular oxygen. These oxygen-starved, unaerated zones of the reaction medium have a tendency to promote undesirable side reactions, rather than the desired oxidation reaction. For example, when para-xylene is partially oxidized to form terephthalic acid, insufficient oxygen availability in the liquid phase of the reaction medium can cause the formation of undesirably high quantities of benzoic acid and coupled aromatic rings, notably including highly undesirable colored molecules known as fluorenones and anthraquinones.

In accordance with one embodiment of the present invention, liquid-phase oxidation is carried out in a bubble column reactor configured and operated in a manner such that the volume fraction of the reaction medium with low gas hold-up values is minimized. This minimization of unaerated zones can be quantified by theoretically partitioning the entire volume of the reaction medium into 2,000 discrete horizontal slices of uniform volume. With the exception of the highest and lowest horizontal slices, each horizontal slice is a discrete volume bounded on its sides by the sidewall of the reactor and bounded on its top and bottom by imaginary horizontal planes. The highest horizontal slice is bounded on its bottom by an imaginary horizontal plane and on its top by the upper surface of the reaction medium. The lowest horizontal slice is bounded on its top by an imaginary horizontal plane and on its bottom by the lower end of the vessel. Once the reaction medium has been theoretically partitioned into 2,000 discrete horizontal slices of equal volume, the time-averaged and volume-averaged gas hold-up of each horizontal slice can be determined. When this method of quantifying the amount of unaerated zones is employed, it is preferred for the number of horizontal slices having a time-averaged and volume-averaged gas hold-up less than 0.1 to be less than 30, more preferably less than 15, still more preferably less than 6, even more preferably less than 4, and most preferably less than 2. It is preferred for the number of horizontal slices having a gas hold-up less than 0.2 to be less than 80, more preferably less than 40, still more preferably less than 20, even more preferably less than 12, and most preferably less than 5. It is preferred for the number of horizontal slices having a gas hold-up less than 0.3 to be less than 120, more preferably less than 80, still more preferably less than 40, even more preferably less than 20, and most preferably less than 15.

Referring again to FIGS. 1 and 2, it has been discovered that positioning oxidant sparger 34 lower in reaction zone 28 provides several advantages, including reduction of the amount of unaerated zones in reaction medium 36. Given a height “H” of reaction medium 36, a length “L” of reaction zone 28, and a maximum diameter “D” of reaction zone 28, it is preferred for a majority (i.e., >50 percent by weight) of the oxidant stream to be introduced into reaction zone 28 within about 0.025 H, 0.022 L, and/or 0.25 D of lower end 52 of reaction zone 28. More preferably, a majority of the oxidant stream is introduced into reaction zone 28 within about 0.02 H, 0.018 L, and/or 0.2 D of lower end 52 of reaction zone 28. Most preferably, a majority of the oxidant stream is introduced into reaction zone 28 within 0.015 H, 0.013 L, and/or 0.15 D of lower end 52 of reaction zone 28.

In the embodiment illustrated in FIG. 2, the vertical distance “Y₁” between lower end 52 of reaction zone 28 and the outlet of upper oxidant openings 68 of oxidant sparger 34 is less than about 0.25 H, 0.022 L, and/or 0.25 D, so that substantially all of the oxidant stream enters reaction zone 28 within about 0.25 H, 0.022 L, and/or 0.25 D of lower end 52 of reaction zone 28. More preferably, Y₁ is less than about 0.02 H, 0.018 L, and/or 0.2 D. Most preferably, Y₁ is less than 0.015 H, 0.013 L, and/or 0.15 D, but more than 0.005 H, 0.004 L, and/or 0.06 D. FIG. 2 illustrates a tangent line 72 at the location where the bottom edge of cylindrical main body 46 of vessel shell 22 joins with the top edge of elliptical lower head 48 of vessel shell 22. Alternatively, lower head 48 can be of any shape, including conical, and the tangent line is still defined as the bottom edge of cylindrical main body 46. The vertical distance “Y₂” between tangent line 72 and the top of oxidant sparger 34 is preferably at least about 0.0012 H, 0.001 L, and/or 0.01 D; more preferably at least about 0.005 H, 0.004 L, and/or 0.05 D; and most preferably at least 0.01 H, 0.008 L, and/or 0.1 D. The vertical distance “Y₃” between lower end 52 of reaction zone 28 and the outlet of lower oxidant openings 70 of oxidant sparger 34 is preferably less than about 0.015 H, 0.013 L, and/or 0.15 D; more preferably less than about 0.012 H, 0.01 L, and/or 0.1 D; and most preferably less than 0.01 H, 0.008 L, and/or 0.075 D, but more than 0.003 H, 0.002 L, and/or 0.025 D.

In a preferred embodiment of the present invention, the openings that discharge the oxidant stream and the feed stream into the reaction zone are configured so that the amount (by weight) of the oxidant or feed stream discharged from an opening is directly proportional to the open area of the opening. Thus, for example, if 50 percent of the cumulative open area defined by all oxidants openings is located within 0.15 D of the bottom of the reaction zone, then 50 weight percent of the oxidant stream enters the reaction zone within 0.15 D of the bottom of the reaction zone and vice-versa.

In addition to the advantages provided by minimizing unaerated zones (i.e., zones with low gas hold-up) in reaction medium 36, it has been discovered that oxidation can be enhanced by maximizing the gas hold-up of the entire reaction medium 36. Reaction medium 36 preferably has time-averaged and volume-averaged gas hold-up of at least about 0.4, more preferably in the range of from about 0.6 to about 0.9, and most preferably in the range of from 0.65 to 0.85. Several physical and operational attributes of bubble column reactor 20 contribute to the high gas hold-up discussed above. For example, for a given reactor size and flow of oxidant stream, the high L:D ratio of reaction zone 28 yields a lower diameter which increases the superficial velocity in reaction medium 36 which in turn increases gas hold-up. Additionally, the actual diameter of a bubble column and the L:D ratio are known to influence the average gas hold-up even for a given constant superficial velocity. In addition, the minimization of unaerated zones, particularly in the bottom of reaction zone 28, contributes to an increased gas hold-up value. Further, the overhead pressure and mechanical configuration of the bubble column reactor can affect operating stability at the high superficial velocities and gas hold-up values disclosed herein.

Furthermore, the inventors have discovered the importance of operating with an optimized overhead pressure to obtain increased gas hold-up and increased mass transfer. It might seem that operating with a lower overhead pressure, which reduces the solubility of molecular oxygen according to a Henry's Law effect, would reduce the mass transfer rate of molecular oxygen from gas to liquid. In a mechanically agitated vessel, such is typically the case because aeration levels and mass transfer rates are dominated by agitator design and overhead pressure. However, in a bubble column reactor according to a preferred embodiment of the present invention, it has been discovered how to use a lower overhead pressure to cause a given mass of gas-phase oxidant stream to occupy more volume, increasing the superficial velocity in reaction medium 36 and in turn increasing the gas hold-up and transfer rate of molecular oxygen.

The balance between bubble coalescence and breakup is an extremely complicated phenomenon, leading on the one hand to a tendency to foam, which reduces internal circulation rates of the liquid phase and which may require very, very large disengaging zones, and on the other hand to a tendency to fewer, very large bubbles that give a lower gas hold-up and lower mass transfer rate from the oxidant stream to the liquid phase. Concerning the liquid phase, its composition, density, viscosity and surface tension, among other factors, are known to interact in a very complicated manner to produce very complicated results even in the absence of a solid-phase. For example, laboratory investigators have found it useful to qualify whether “water” is tap water, distilled water, or de-ionized water, when reporting and evaluating observations for even simple water-air bubble columns. For complex mixtures in the liquid phase and for the addition of a solid phase, the degree of complexity rises further. The surface irregularities of individual particles of solids, the average size of solids, the particle size distribution, the amount of solids relative to the liquid phase, and the ability of the liquid to wet the surface of the solid, among other things, are all important in their interaction with the liquid phase and the oxidant stream in establishing what bubbling behavior and natural convection flow patterns will result.

Thus, the ability of the bubble column reactor to function usefully with the high superficial velocities and high gas hold-up disclosed herein depends, for example, on an appropriate selection of: (1) the composition of the liquid phase of the reaction medium; (2) the amount and type of precipitated solids, both of which can be adjusted by reaction conditions; (3) the amount of oxidant stream fed to the reactor; (4) the overhead pressure, which affects the volumetric flow of oxidant stream, the stability of bubbles, and, via the energy balance, the reaction temperature; (5) the reaction temperature itself, which affects the fluid properties, the properties of precipitated solids, and the specific volume of the oxidant stream; and (6) the geometry and mechanical details of the reaction vessel, including the L:D ratio.

Referring again to FIG. 1, it has been discovered that improved distribution of the oxidizable compound (e.g., para-xylene) in reaction medium 36 can be provided by introducing the liquid-phase feed stream into reaction zone 28 at multiple vertically-spaced locations. Preferably, the liquid-phase feed stream is introduced into reaction zone 28 via at least 3 feed openings, more preferably at least 4 feed openings. As used herein, the term “feed openings” shall denote openings where the liquid-phase feed stream is discharged into reaction zone 28 for mixing with reaction medium 36. It is preferred for at least 2 of the feed openings to be vertically-spaced from one another by at least about 0.5 D, more preferably at least about 1.5 D, and most preferably at least 3 D. However, it is preferred for the highest feed opening to be vertically-spaced from the lowest oxidant opening by not more than about 0.75 H, 0.65 L, and/or 8 D; more preferably not more than about 0.5 H, 0.4 L, and/or 5 D; and most preferably not more than 0.4 H, 0.35 L, and/or 4 D.

Although it is desirable to introduce the liquid-phase feed stream at multiple vertical locations, it has also been discovered that improved distribution of the oxidizable compound in reaction medium 36 is provided if the majority of the liquid-phase feed stream is introduced into the bottom half of reaction medium 36 and/or reaction zone 28. Preferably, at least about 75 weight percent of the liquid-phase feed stream is introduced into the bottom half of reaction medium 36 and/or reaction zone 28. Most preferably, at least 90 weight percent of the liquid-phase feed stream is introduced into the bottom half of reaction medium 36 and/or reaction zone 28. In addition, it is preferred for at least about 30 weight percent of the liquid-phase feed stream to be introduced into reaction zone 28 within about 1.5 D of the lowest vertical location where the oxidant stream is introduced into reaction zone 28. This lowest vertical location where the oxidant stream is introduced into reaction zone 28 is typically at the bottom of oxidant sparger; however, a variety of alternative configurations for introducing the oxidant stream into reaction zone 28 are contemplated by a preferred embodiment of the present invention. Preferably, at least about 50 weight percent of the liquid-phase feed is introduced within about 2.5 D of the lowest vertical location where the oxidant stream is introduced into reaction zone 28. Preferably, at least about 75 weight percent of the liquid-phase feed stream is introduced within about 5 D of the lowest vertical location where the oxidant stream is introduced into reaction zone 28.

Each feed opening defines an open area through which the feed is discharged. It is preferred that at least about 30 percent of the cumulative open area of all the feed inlets is located within about 1.5 D of the lowest vertical location where the oxidant stream is introduced into reaction zone 28. Preferably, at least about 50 percent of the cumulative open area of all the feed inlets is located within about 2.5 D of the lowest vertical location where the oxidant stream is introduced into reaction zone 28. Preferably, at least about 75 percent of the cumulative open area of all the feed inlets is located within about 5 D of the lowest vertical location where the oxidant stream is introduced into reaction zone 28.

Referring again to FIG. 1, in one embodiment of the present invention, feed inlets 32a,b,c,d are simply a series of vertically-aligned openings along one side of vessel shell 22. These feed openings preferably have substantially similar diameters of less than about 7 centimeters, more preferably in the range of from about 0.25 to about 5 centimeters, and most preferably in the range of from 0.4 to 2 centimeters. Bubble column reactor 20 is preferably equipped with a system for controlling the flow rate of the liquid-phase feed stream out of each feed opening. Such flow control system preferably includes an individual flow control valve 74a,b,c,d for each respective feed inlet 32a,b,c,d. In addition, it is preferred for bubble column reactor 20 to be equipped with a flow control system that allows at least a portion of the liquid-phase feed stream to be introduced into reaction zone 28 at an elevated inlet superficial velocity of at least about 2 meters per second, more preferably at least about 5 meters per second, still more preferably at least about 6 meters per second, and most preferably in the range of from 8 to 20 meters per second. As used herein, the term “inlet superficial velocity” denotes the time-averaged volumetric flow rate of the feed stream out of the feed opening divided by the area of the feed opening. Preferably, at least about 50 weight percent of the feed stream is introduced into reaction zone 28 at an elevated inlet superficial velocity. Most preferably, substantially all the feed stream is introduced into reaction zone 28 at an elevated inlet superficial velocity.

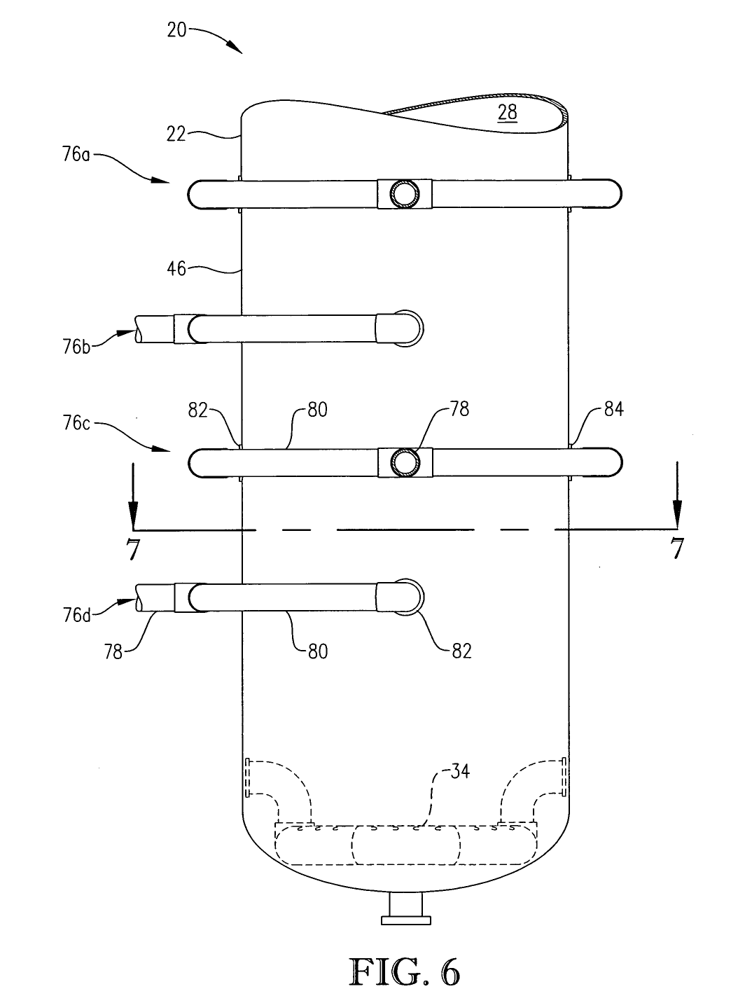

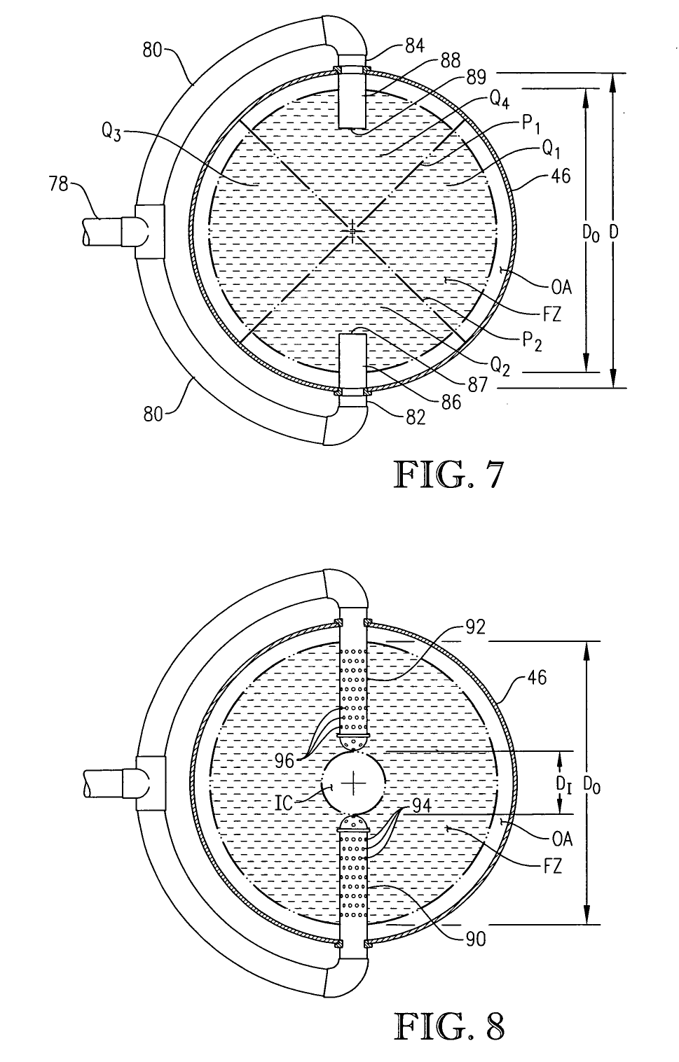

Referring now to FIGS. 6-7, an alternative system for introducing the liquid-phase feed stream into reaction zone 28 is illustrated. In this embodiment, the feed stream is introduced into reaction zone 28 at four different elevations. Each elevation is equipped with a respective feed distribution system 76a,b,c,d. Each feed distribution system 76 includes a main feed conduit 78 and a manifold 80. Each manifold 80 is provided with at least two outlets 82,84 coupled to respective insert conduits 86,88, which extend into reaction zone 28 of vessel shell 22. Each insert conduit 86,88 presents a respective feed opening 87,89 for discharging the feed stream into reaction zone 28. Feed openings 87,89 preferably have substantially similar diameters of less than about 7 centimeters, more preferably in the range of from about 0.25 to about 5 centimeters, and most preferably in the range of from 0.4 to 2 centimeters. It is preferred for feed openings 87,89 of each feed distribution system 76a,b,c,d to be diametrically opposed so as to introduce the feed stream into reaction zone 28 in opposite directions. Further, it is preferred for the diametrically opposed feed openings 86,88 of adjacent feed distribution systems 76 to be oriented at 90 degrees of rotation relative to one another. In operation, the liquid-phase feed stream is charged to main feed conduit 78 and subsequently enters manifold 80. Manifold 80 distributes the feed stream evenly for simultaneous introduction on opposite sides of reactor 20 via feed openings 87,89.

FIG. 8 illustrates an alternative configuration wherein each feed distribution system 76 is equipped with bayonet tubes 90,92 rather than insert conduits 86,88 (shown in FIG. 7). Bayonet tubes 90,92 project into reaction zone 28 and include a plurality of small feed openings 94,96 for discharging the liquid-phase feed into reaction zone 28. It is preferred for the small feed openings 94,96 of bayonet tubes 90,92 to have substantially the same diameters of less than about 50 millimeters, more preferably about 2 to about 25 millimeters, and most preferably 4 to 15 millimeters.

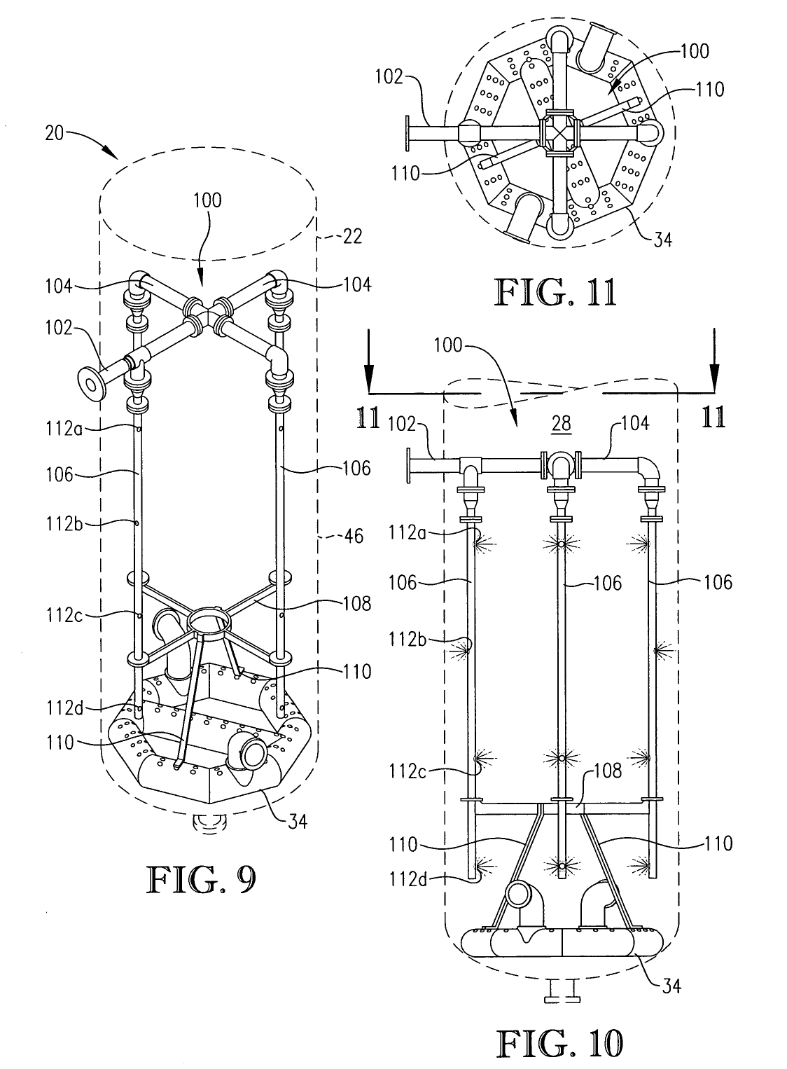

FIGS. 9-11 illustrate an alternative feed distribution system 100. Feed distribution system 100 introduces the liquid-phase feed stream at a plurality of vertically-spaced and laterally-spaced locations without requiring multiple penetrations of the sidewall of bubble column reactor 20. Feed introduction system 100 generally includes a single inlet conduit 102, a header 104, a plurality of upright distribution tubes 106, a lateral support mechanism 108, and a vertical support mechanism 110. Inlet conduit 102 penetrates the sidewall of main body 46 of vessel shell 22. Inlet conduit 102 is fluidly coupled to header 104. Header 104 distributes the feed stream received from inlet conduit 102 evenly among upright distribution tubes 106. Each distribution tube 106 has a plurality of vertically-spaced feed openings 112a,b,c,d for discharging the feed stream into reaction zone 28. Lateral support mechanism 108 is coupled to each distribution tube 106 and inhibits relative lateral movement of distribution tubes 106. Vertical support mechanism 110 is preferably coupled to lateral support mechanism 108 and to the top of oxidant sparger 34. Vertical support mechanism 110 substantially inhibits vertical movement of distribution tubes 106 in reaction zone 28. It is preferred for feed openings 112 to have substantially the same diameters of less than about 50 millimeters, more preferably about 2 to about 25 millimeters, and most preferably 4 to 15 millimeters. The vertical spacing of feed openings 112 of feed distribution system 100 illustrated in FIGS. 9-11 can be substantially the same as described above with reference to the feed distribution system of FIG. 1.

It has been discovered that the flow patterns of the reaction medium in many bubble column reactors can permit uneven azimuthal distribution of the oxidizable compound in the reaction medium, especially when the oxidizable compound is primarily introduced along one side of the reaction medium. As used herein, the term “azimuthal” shall denote an angle or spacing around the upright axis of elongation of the reaction zone. As used herein, “upright” shall mean within 45° of vertical. In one embodiment of the present invention, the feed stream containing the oxidizable compound (e.g., para-xylene) is introduced into the reaction zone via a plurality of azimuthally-spaced feed openings. These azimuthally-spaced feed openings can help prevent regions of excessively high and excessively low oxidizable compound concentrations in the reaction medium. The various feed introduction systems illustrated in FIGS. 6-11 are examples of systems that provide proper azimuthal spacing of feed openings.

Referring again to FIG. 7, in order to quantify the azimuthally-spaced introduction of the liquid-phase feed stream into the reaction medium, the reaction medium can be theoretically partitioned into four upright azimuthal quadrants “Q₁,Q₂,Q₃,Q₄” of approximately equal volume. These azimuthal quadrants “Q₁,Q₂,Q₃,Q₄” are defined by a pair of imaginary intersecting perpendicular vertical planes “P₁,P₂” extending beyond the maximum vertical dimension and maximum radial dimension of the reaction medium. When the reaction medium is contained in a cylindrical vessel, the line of intersection of the imaginary intersecting vertical planes P₁,P₂ will be approximately coincident with the vertical centerline of the cylinder, and each azimuthal quadrant Q₁,Q₂,Q₃,Q₄ will be a generally wedge-shaped vertical volume having a height equal to the height of the reaction medium. It is preferred for a substantial portion of the oxidizable compound to be discharged into the reaction medium via feed openings located in at least two different azimuthal quadrants.

In a preferred embodiment of the present invention, not more than about 80 weight percent of the oxidizable compound is discharged into the reaction medium through feed openings that can be located in a single azimuthal quadrant. More preferably, not more than about 60 weight percent of the oxidizable compound is discharged into the reaction medium through feed openings that can be located in a single azimuthal quadrant. Most preferably, not more than 40 weight percent of the oxidizable compound is discharged into the reaction medium through feed openings that can be located in a single azimuthal quadrant. These parameters for azimuthal distribution of the oxidizable compound are measured when the azimuthal quadrants are azimuthally oriented such that the maximum possible amount of oxidizable compound is being discharged into one of the azimuthal quadrants. For example, if the entire feed stream is discharged into the reaction medium via two feed openings that are azimuthally spaced from one another by 89 degrees, for purposes of determining azimuthal distribution in four azimuthal quadrants, 100 weight percent of the feed stream is discharged into the reaction medium in a single azimuthal quadrant because the azimuthal quadrants can be azimuthally oriented in such a manner that both of the feed openings are located in a single azimuthal quadrant.

In addition to the advantages associated with the proper azimuthal-spacing of the feed openings, it has also been discovered that proper radial spacing of the feed openings in a bubble column reactor can also be important. It is preferred for a substantial portion of the oxidizable compound introduced into the reaction medium to be discharged via feed openings that are radially spaced inwardly from the sidewall of the vessel. Thus, in one embodiment of the present invention, a substantial portion of the oxidizable compound enters the reaction zone via feed openings located in a “preferred radial feed zone” that is spaced inwardly from the upright sidewalls defining the reaction zone.

Referring again to FIG. 7, the preferred radial feed zone “FZ” can take the shape of a theoretical upright cylinder centered in reaction zone 28 and having an outer diameter “D_{O}38 of 0.9 D, where “D” is the diameter of reaction zone 28. Thus, an outer annulus “OA” having a thickness of 0.05 D is defined between the preferred radial feed zone FZ and the inside of the sidewall defining reaction zone 28. It is preferred for little or none of the oxidizable compound to be introduced into reaction zone 28 via feed openings located in this outer annulus OA.

In another embodiment, it is preferred for little or none of the oxidizable compound to be introduced into the center of reaction zone 28. Thus, as illustrated in FIG. 8, the preferred radial feed zone FZ can take the shape of a theoretical upright annulus centered in reaction zone 28, having an outer diameter D_{O} of 0.9 D, and having an inner diameter D_{I} of 0.2 D. Thus, in this embodiment, an inner cylinder IC having a diameter of 0.2 D is “cut out” of the center of the preferred radial feed zone FZ. It is preferred for little or none of the oxidizable compound to be introduced into reaction zone 28 via feed openings located in this inner cylinder IC.

In a preferred embodiment of the present invention, a substantial portion of the oxidizable compound is introduced into reaction medium 36 via feed openings located in the preferred radial feed zone, regardless of whether the preferred radial feed zone has the cylindrical or annular shape described above. More preferably, at least about 25 weight percent of the oxidizable compound is discharged into reaction medium 36 via feed openings located in the preferred radial feed zone. Still more preferably, at least about 50 weight percent of the oxidizable compound is discharged into reaction medium 36 via feed openings located in the preferred radial feed zone. Most preferably, at least 75 weight percent of the oxidizable compound is discharged into reaction medium 36 via feed openings located in the preferred radial feed zone.

Although the theoretical azimuthal quadrants and theoretical preferred radial feed zone illustrated in FIGS. 7 and 8 are described with reference to the distribution of the liquid-phase feed stream, it has been discovered that proper azimuthal and radial distribution of the gas-phase oxidant stream can also provide certain advantages. Thus, in one embodiment of the present invention, the description of the azimuthal and radial distribution of the liquid-phase feed stream, provided above, also applies to the manner in which the gas-phase oxidant stream is introduced into the reaction medium 36.

As mentioned above, certain physical and operational features of bubble column reactor 20, described above with reference to FIGS. 1-11, provide for vertical gradients in the pressure, temperature, and reactant (i.e., oxygen and oxidizable compound) concentrations of reaction medium 36. As discussed above, these vertical gradients can provide for a more effective and economical oxidation process as compared to conventional oxidations processes, which favor a well-mixed reaction medium of relatively uniform pressure, temperature, and reactant concentration throughout. The vertical gradients for oxygen, oxidizable compound (e.g., para-xylene), and temperature made possible by employing an oxidation system in accordance with an embodiment of the present invention will now be discussed in greater detail.

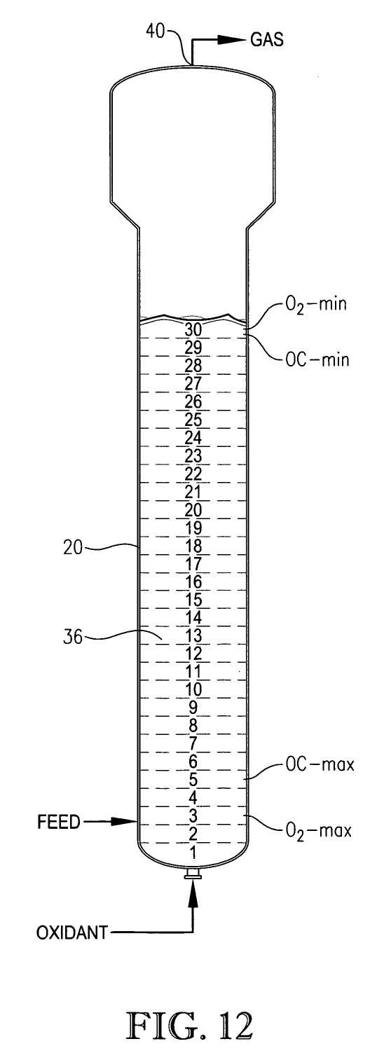

Referring now to FIG. 12, in order to quantify the reactant concentration gradients existing in reaction medium 36 during oxidation in bubble column reactor 20, the entire volume of reaction medium 36 can be theoretically partitioned into 30 discrete horizontal slices of equal volume. FIG. 12 illustrates the concept of dividing reaction medium 36 into 30 discrete horizontal slices of equal volume. With the exception of the highest and lowest horizontal slices, each horizontal slice is a discrete volume bounded on its top and bottom by imaginary horizontal planes and bounded on its sides by the wall of reactor 20. The highest horizontal slice is bounded on its bottom by an imaginary horizontal plane and on its top by the upper surface of reaction medium 36. The lowest horizontal slice is bounded on its top by an imaginary horizontal plane and on its bottom by the bottom of the vessel shell. Once reaction medium 36 has been theoretically partitioned into 30 discrete horizontal slices of equal volume, the time-averaged and volume-averaged concentration of each horizontal slice can then be determined. The individual horizontal slice having the maximum concentration of all 30 horizontal slices can be identified as the “C-max horizontal slice.” The individual horizontal slice located above the C-max horizontal slice and having the minimum concentration of all horizontal slices located above the C-max horizontal slice can be identified as the “C-min horizontal slice.” The vertical concentration gradient can then be calculated as the ratio of the concentration in the C-max horizontal slice to the concentration in the C-min horizontal slice.

With respect to quantifying the oxygen concentration gradient, when reaction medium 36 is theoretically partitioned into 30 discrete horizontal slices of equal volume, an O₂-max horizontal slice is identified as having the maximum oxygen concentration of all the 30 horizontal slices and an O₂-min horizontal slice is identified as having the minimum oxygen concentration of the horizontal slices located above the O₂-max horizontal slice. The oxygen concentrations of the horizontal slices are measured in the gas phase of reaction medium 36 on a time-averaged and volume-averaged molar wet basis. It is preferred for the ratio of the oxygen concentration of the O₂-max horizontal slice to the oxygen concentration of the O₂-min horizontal slice to be in the range of from about 2:1 to about 25:1, more preferably in the range of from about 3:1 to about 15:1, and most preferably in the range of from 4:1 to 10:1.

Typically, the O₂-max horizontal slice will be located near the bottom of reaction medium 36, while the O₂-min horizontal slice will be located near the top of reaction medium 36. Preferably, the O₂-min horizontal slice is one of the 5 upper-most horizontal slices of the 30 discrete horizontal slices. Most preferably, the O₂-min horizontal slice is the upper-most one of the 30 discrete horizontal slices, as illustrated in FIG. 12. Preferably, the O₂-max horizontal slice is one of the 10 lower-most horizontal slices of the 30 discrete horizontal slices. Most preferably, the O₂-max horizontal slice is one of the 5 lower-most horizontal slices of the 30 discrete horizontal slices. For example, FIG. 12 illustrates the O₂-max horizontal slice as the third horizontal slice from the bottom of reactor 20. It is preferred for the vertical spacing between the O₂-min and O₂-max horizontal slices to be at least about 2 W, more preferably at least about 4 W, and most preferably at least 6 W. It is preferred for the vertical spacing between the O₂-min and O₂-max horizontal slices to be at least about 0.2 H, more preferably at least about 0.4 H, and most preferably at least 0.6 H

The time-averaged and volume-averaged oxygen concentration, on a wet basis, of the O₂-min horizontal slice is preferably in the range of from about 0.1 to about 3 mole percent, more preferably in the range of from about 0.3 to about 2 mole percent, and most preferably in the range of from 0.5 to 1.5 mole percent. The time-averaged and volume-averaged oxygen concentration of the O₂-max horizontal slice is preferably in the range of from about 4 to about 20 mole percent, more preferably in the range of from about 5 to about 15 mole percent, and most preferably in the range of from 6 to 12 mole percent. The time-averaged concentration of oxygen, on a dry basis, in the gaseous effluent discharged from reactor 20 via gas outlet 40 is preferably in the range of from about 0.5 to about 9 mole percent, more preferably in the range of from about 1 to about 7 mole percent, and most preferably in the range of from 1.5 to 5 mole percent.

Because the oxygen concentration decays so markedly toward the top of reaction medium 36, it is desirable that the demand for oxygen be reduced in the top of reaction medium 36. This reduced demand for oxygen near the top of reaction medium 36 can be accomplished by creating a vertical gradient in the concentration of the oxidizable compound (e.g., para-xylene), where the minimum concentration of oxidizable compound is located near the top of reaction medium 36.

With respect to quantifying the oxidizable compound (e.g., para-xylene) concentration gradient, when reaction medium 36 is theoretically partitioned into 30 discrete horizontal slices of equal volume, an OC-max horizontal slice is identified as having the maximum oxidizable compound concentration of all the 30 horizontal slices and an OC-min horizontal slice is identified as having the minimum oxidizable compound concentration of the horizontal slices located above the OC-max horizontal slice. The oxidizable compound concentrations of the horizontal slices are measured in the liquid phase on a time-averaged and volume-averaged mass fraction basis. It is preferred for the ratio of the oxidizable compound concentration of the OC-max horizontal slice to the oxidizable compound concentration of the OC-min horizontal slice to be greater than about 5:1, more preferably greater than about 10:1, still more preferably greater than about 20:1, and most preferably in the range of from 40:1 to 1000:1.

Typically, the OC-max horizontal slice will be located near the bottom of reaction medium 36, while the OC-min horizontal slice will be located near the top of reaction medium 36. Preferably, the OC-min horizontal slice is one of the 5 upper-most horizontal slices of the 30 discrete horizontal slices. Most preferably, the OC-min horizontal slice is the upper-most one of the 30 discrete horizontal slices, as illustrated in FIG. 12. Preferably, the OC-max horizontal slice is one of the 10 lower-most horizontal slices of the 30 discrete horizontal slices. Most preferably, the OC-max horizontal slice is one of the 5 lower-most horizontal slices of the 30 discrete horizontal slices. For example, FIG. 12 illustrates the OC-max horizontal slice as the fifth horizontal slice from the bottom of reactor 20. It is preferred for the vertical spacing between the OC-min and OC-max horizontal slices to be at least about 2 W, where “W” is the maximum width of reaction medium 36. More preferably, the vertical spacing between the OC-min and OC-max horizontal slices is at least about 4 W, and most preferably at least 6 W. Given a height “H” of reaction medium 36, it is preferred for the vertical spacing between the OC-min and OC-max horizontal slices to be at least about 0.2 H, more preferably at least about 0.4 H, and most preferably at least 0.6 H.

The time-averaged and volume-averaged oxidizable compound (e.g., para-xylene) concentration in the liquid phase of the OC-min horizontal slice is preferably less than about 5,000 ppmw, more preferably less than about 2,000 ppmw, still more preferably less than about 400 ppmw, and most preferably in the range of from 1 ppmw to 100 ppmw. The time-averaged and volume-averaged oxidizable compound concentration in the liquid phase of the OC-max horizontal slice is preferably in the range of from about 100 ppmw to about 10,000 ppmw, more preferably in the range of from about 200 ppmw to about 5,000 ppmw, and most preferably in the range of from 500 ppmw to 3,000 ppmw.

Although it is preferred for bubble column reactor 20 to provide vertical gradients in the concentration of the oxidizable compound, it is also preferred that the volume percent of reaction medium 36 having an oxidizable compound concentration in the liquid phase above 1,000 ppmw be minimized. Preferably, the time-averaged volume percent of reaction medium 36 having an oxidizable compound concentration in the liquid phase above 1,000 ppmw is less than about 9 percent, more preferably less than about 6 percent, and most preferably less than 3 percent. Preferably, the time-averaged volume percent of reaction medium 36 having an oxidizable compound concentration in the liquid phase above 2,500 ppmw is less than about 1.5 percent, more preferably less than about 1 percent, and most preferably less than 0.5 percent. Preferably, the time-averaged volume percent of reaction medium 36 having an oxidizable compound concentration in the liquid phase above 10,000 ppmw is less than about 0.3 percent, more preferably less than about 0.1 percent, and most preferably less than 0.03 percent. Preferably, the time-averaged volume percent of reaction medium 36 having an oxidizable compound concentration in the liquid phase above 25,000 ppmw is less than about 0.03 percent, more preferably less than about 0.015 percent, and most preferably less than 0.007 percent. The inventors note that the volume of reaction medium 36 having the elevated levels of oxidizable compound need not lie in a single contiguous volume. At many times, the chaotic flow patterns in a bubble column reaction vessel produce simultaneously two or more continuous but segregated portions of reaction medium 36 having the elevated levels of oxidizable compound. At each time used in the time averaging, all such continuous but segregated volumes larger than 0.0001 volume percent of the total reaction medium are added together to determine the total volume having the elevated levels of oxidizable compound concentration in the liquid phase.

It is now noted that many of the inventive features described herein can be employed in multiple oxidation reactor systems—not just systems employing a single oxidation reactor. In addition, certain inventive features described herein can be employed in mechanically-agitated and/or flow-agitated oxidation reactors—not just bubble-agitated reactors (i.e., bubble column reactors). For example, the inventors have discovered certain advantages associated with staging/varying oxygen concentration and/or oxygen consumption rate throughout the reaction medium. The advantages realized by the staging of oxygen concentration/consumption in the reaction medium can be realized whether the total volume of the reaction medium is contained in a single vessel or in multiple vessels. Further, the advantages realized by the staging of oxygen concentration/consumption in the reaction medium can be realized whether the reaction vessel(s) is mechanically-agitated, flow-agitated, and/or bubble-agitated.

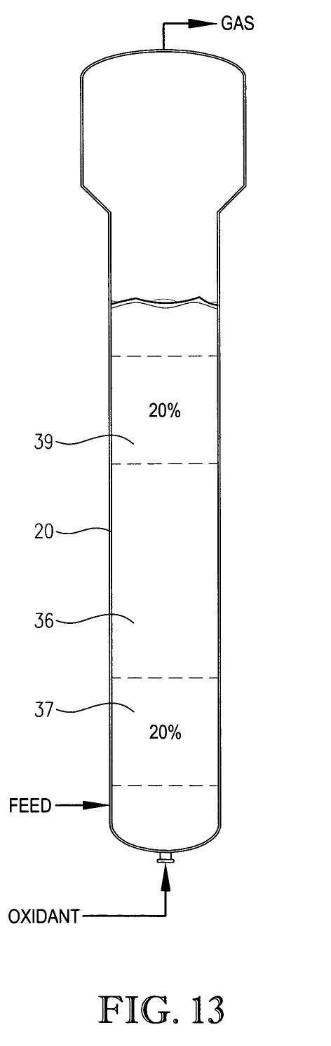

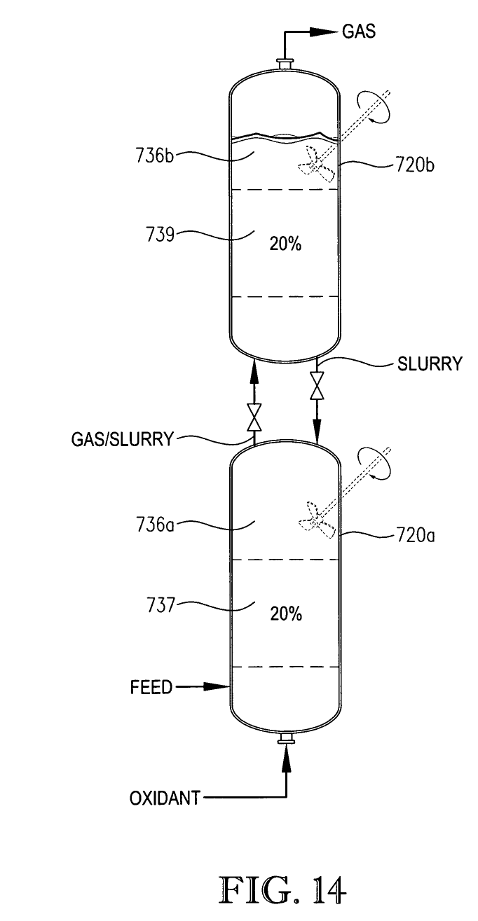

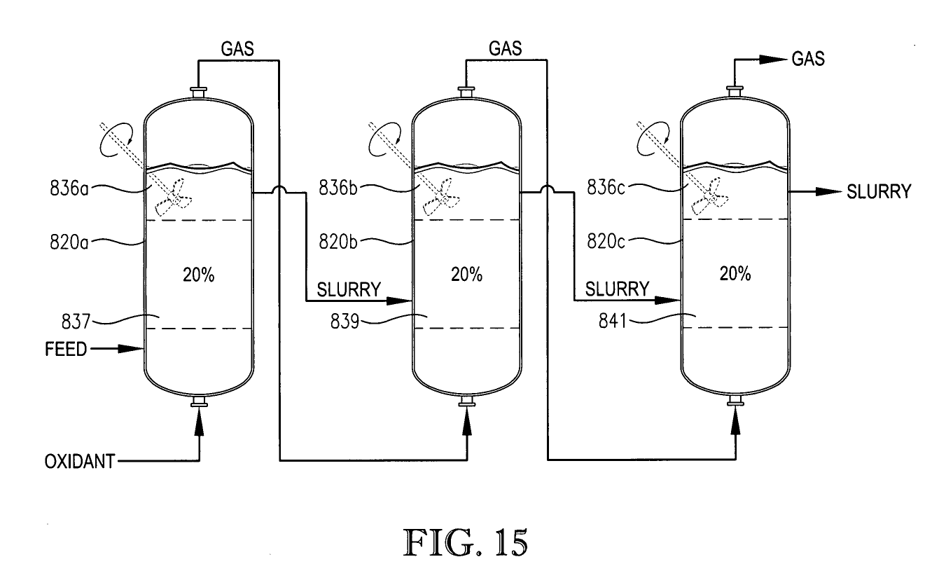

One way of quantifying the degree of staging of oxygen concentration and/or consumption rate in a reaction medium is to compare two or more distinct 20-percent continuous volumes of the reaction medium. These 20-percent continuous volumes need not be defined by any particular shape. However, each 20-percent continuous volume must be formed of a contiguous volume of the reaction medium (i.e., each volume is “continuous”), and the 20-percent continuous volumes must not overlap one another (i.e., the volumes are “distinct”). FIGS. 13-15 illustrate that these distinct 20-percent continuous volumes can be located in the same reactor (FIG. 13) or in multiple reactors (FIGS. 14 and 15). It is noted that the reactors illustrated in FIGS. 13-15 can be mechanically-agitated, flow-agitated, and/or bubble-agitated reactors. In one embodiment, it is preferred for the reactors illustrated in FIGS. 13-15 to be bubble-agitated reactors (i.e., bubble column reactors).

Referring now to FIG. 13, reactor 20 is illustrated as containing a reaction medium 36. Reaction medium 36 includes a first distinct 20-percent continuous volume 37 and a second distinct 20-percent continuous volume 39.

Referring now to FIG. 14, a multiple reactor system is illustrated as including a first reactor 720a and a second reactor 720b. Reactors 720a,b, cooperatively contain a total volume of a reaction medium 736. First reactor 720a contains a first reaction medium portion 736a, while second reactor 720b contains a second reaction medium portion 736b. A first distinct 20-percent continuous volume 737 of reaction medium 736 is shown as being defined within first reactor 720a, while a second distinct 20-percent continuous volume 739 of reaction medium 736 is shown as being defined within second reactor 720b.

Referring now to FIG. 15, a multiple reactor system is illustrated as including a first reactor 820a, a second reactor 820b, and a third reactor 820c. Reactors 820a,b,c cooperatively contain a total volume of a reaction medium 836. First reactor 820a contains a first reaction medium portion 836a; second reactor 820b contains a second reaction medium portion 836b; and third reactor 820c contains a third reaction medium portion 836c. A first distinct 20-percent continuous volume 837 of reaction medium 836 is shown as being defined within first reactor 820a; a second distinct 20-percent continuous volume 839 of reaction medium 836 is shown as being defined within second reactor 820b; and a third distinct 20-percent continuous volume 841 of reaction medium 836 is show as being defined within third reactor 820c.

The staging of oxygen availability in the reaction medium can be quantified by referring to the 20-percent continuous volume of reaction medium having the most abundant mole fraction of oxygen in the gas phase and by referring to the 20-percent continuous volume of reaction medium having the most depleted mole fraction of oxygen in the gas phase. In the gas phase of the distinct 20-percent continuous volume of the reaction medium containing the highest concentration of oxygen in the gas phase, the time-averaged and volume-averaged oxygen concentration, on a wet basis, is preferably in the range of from about 3 to about 18 mole percent, more preferably in the range of from about 3.5 to about 14 mole percent, and most preferably in the range of from 4 to 10 mole percent. In the gas phase of the distinct 20-percent continuous volume of the reaction medium containing the lowest concentration of oxygen in the gas phase, the time-averaged and volume-averaged oxygen concentration, on a wet basis, is preferably in the range of from about 0.3 to about 5 mole percent, more preferably in the range of from about 0.6 to about 4 mole percent, and most preferably in the range of from 0.9 to 3 mole percent. Furthermore, the ratio of the time-averaged and volume-averaged oxygen concentration, on a wet basis, in the most abundant 20-percent continuous volume of reaction medium compared to the most depleted 20-percent continuous volume of reaction medium is preferably in the range of from about 1.5:1 to about 20:1, more preferably in the range of from about 2:1 to about 12:1, and most preferably in the range of from 3:1 to 9:1.

The staging of oxygen consumption rate in the reaction medium can be quantified in terms of an oxygen-STR, initially described above. Oxygen-STR was previously describe in a global sense (i.e., from the perspective of the average oxygen-STR of the entire reaction medium); however, oxygen-STR may also be considered in a local sense (i.e., a portion of the reaction medium) in order to quantify staging of the oxygen consumption rate throughout the reaction medium.

The inventors have discovered that it is very useful to cause the oxygen-STR to vary throughout the reaction medium in general harmony with the desirable gradients disclosed herein relating to pressure in the reaction medium and to the mole fraction of molecular oxygen in the gas phase of the reaction medium. Thus, it is preferable that the ratio of the oxygen-STR of a first distinct 20-percent continuous volume of the reaction medium compared to the oxygen-STR of a second distinct 20-percent continuous volume of the reaction medium be in the range of from about 1.5:1 to about 20:1, more preferably in the range of from about 2:1 to about 12:1, and most preferably in the range of from 3:1 to 9:1. In one embodiment the “first distinct 20-percent continuous volume” is located closer than the “second distinct 20-percent continuous volume” to the location where molecular oxygen is initially introduced into the reaction medium. These large gradients in oxygen-STR are desirable whether the partial oxidation reaction medium is contained in a bubble column oxidation reactor or in any other type of reaction vessel in which gradients are created in pressure and/or mole fraction of molecular oxygen in the gas phase of the reaction medium (e.g., in a mechanically agitated vessel having multiple, vertically disposed stirring zones achieved by using multiple impellers having strong radial flow, possibly augmented by generally horizontal baffle assemblies, with oxidant flow rising generally upwards from a feed near the lower portion of the reaction vessel, notwithstanding that considerable back-mixing of oxidant flow may occur within each vertically disposed stirring zone and that some back-mixing of oxidant flow may occur between adjacent vertically disposed stirring zones). That is, when a gradient exists in the pressure and/or mole fraction of molecular oxygen in the gas phase of the reaction medium, the inventors have discovered that it is desirable to create a similar gradient in the chemical demand for dissolved oxygen by the means disclosed herein.

A preferred means of causing the local oxygen-STR to vary is by controlling the locations of feeding the oxidizable compound and by controlling the mixing of the liquid phase of the reaction medium to control gradients in concentration of oxidizable compound according to other disclosures of the present invention. Other useful means of causing the local oxygen-STR to vary include causing variation in reaction activity by causing local temperature variation and by changing the local mixture of catalyst and solvent components (e.g., by introducing an additional gas to cause evaporative cooling in a particular portion of the reaction medium and by adding a solvent stream containing a higher amount of water to decrease activity in a particular portion of the reaction medium).

As discussed above with reference to FIGS. 14 and 15, the partial oxidation reaction can be usefully conducted in multiple reaction vessels wherein at least a portion, preferably at least 25 percent, more preferably at least 50 percent, and most preferable at least 75 percent, of the molecular oxygen exiting from a first reaction vessel is conducted to one or more subsequent reaction vessels for consumption of an additional increment, preferably more than 10 percent, more preferably more than 20 percent, and most preferably more than 40 percent, of the molecular oxygen exiting the first/upstream reaction vessel. When using such a series flow of molecular oxygen from one reactor to others, it is desirable that the first reaction vessel is operated with a higher reaction intensity than at least one of the subsequent reaction vessels, preferably with the ratio of the vessel-average-oxygen-STR within the first reaction vessel to the vessel-average-oxygen-STR within the subsequent reaction vessel in the range of from about 1.5:1 to about 20:1, more preferably in the range of from about 2:1 to about 12:1, and most preferably in the range of from 3:1 to 9:1.

As discussed above, all types of first reaction vessel (e.g.; bubble column, mechanically-agitated, back-mixed, internally staged, plug flow, and so on) and all types of subsequent reaction vessels, which may or not be of different type than the first reaction vessel, are useful for series flow of molecular oxygen to subsequent reaction vessels with according to the present invention. The means of causing the vessel-average-oxygen-STR to decline within subsequent reaction vessels usefully include reduced temperature, reduced concentrations of oxidizable compound, and reduced reaction activity of the particular mixture of catalytic components and solvent (e.g., reduced concentration of cobalt, increased concentration of water, and addition of a catalytic retardant such as small quantities of ionic copper).

In flowing from the first reaction vessel to a subsequent reaction vessel, the oxidant stream may be treated by any means known in the art such as compression or pressure reduction, cooling or heating, and removing mass or adding mass of any amount or any type. However, the use of declining vessel-average-oxygen-STR in subsequent reaction vessels is particularly useful when the absolute pressure in the upper portion of the first reaction vessel is less than about 2.0 megapascal, more preferably less than about 1.6 megapascal, and most preferably less than 1.2 megapascal. Furthermore, the use of declining vessel-average-oxygen-STR in subsequent reaction vessels is particularly useful when the ratio of the absolute pressure in the upper portion of the first reaction vessel compared to the absolute pressure in the upper portion of at least one subsequent reaction vessel is in the range from about 0.5:1 to 6:1, more preferably in a range from about 0.6:1 to about 4:1, and most preferably in a range from 0.7:1 to 2:1. Pressure reductions in subsequent vessels below these lower bounds overly reduce the availability of molecular oxygen, and pressure increases above these upper bounds are increasingly costly compared to using a fresh supply of oxidant.

When using series flow of molecular oxygen to subsequent reaction vessels having declining vessel-average-oxygen-STR, fresh feed streams of oxidizable compound, solvent and oxidant may flow into subsequent reaction vessels and/or into the first reaction vessel. Flows of the liquid phase and the solid phase, if present, of the reaction medium may flow in any direction between reaction vessels. All or part of the gas phase leaving the first reaction vessel and entering a subsequent reaction vessel may flow separated from or commingled with portions of the liquid phase or the solid phase, if present, of the reaction medium from the first reaction vessel. A flow of product stream comprising liquid phase and solid phase, if present, may be withdrawn from the reaction medium in any reaction vessel in the system.

Referring again to FIGS. 1-15, oxidation is preferably carried out in bubble column reactor 20 under conditions that are markedly different, according to preferred embodiments disclosed herein, than conventional oxidation reactors. When bubble column reactor 20 is used to carry out the liquid-phase partial oxidation of para-xylene to crude terephthalic acid (CTA) according to preferred embodiments disclosed herein, the spatial profiles of local reaction intensity, of local evaporation intensity, and of local temperature combined with the liquid flow patterns within the reaction medium and the preferred, relatively low oxidation temperatures contribute to the formation of CTA particles having unique and advantageous properties.

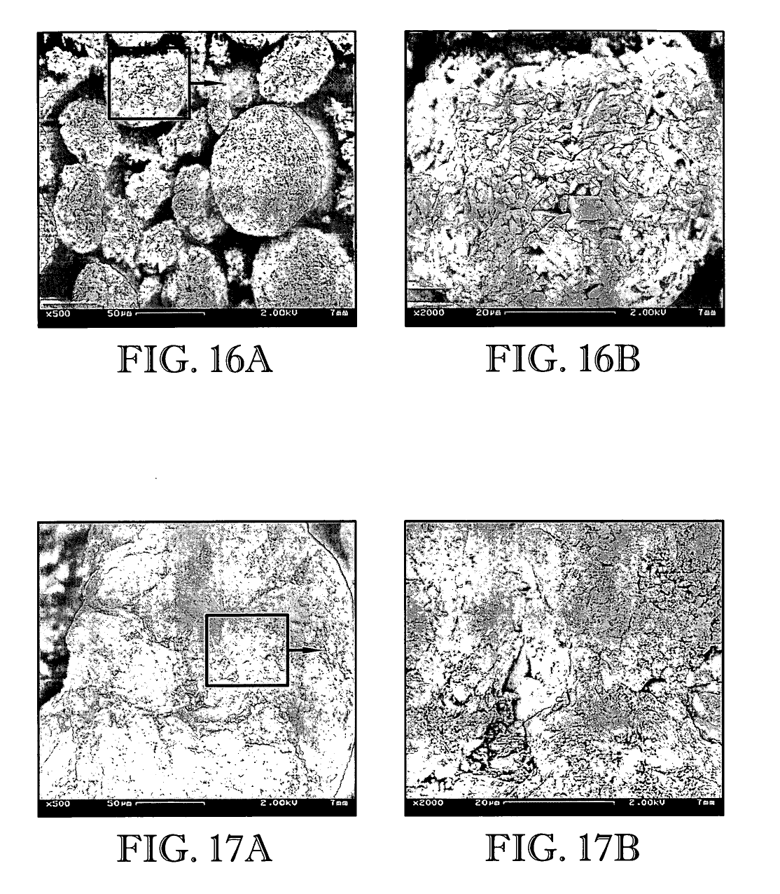

FIGS. 16A and 16B illustrate base CTA particles produced in accordance with one embodiment of the present invention. FIG. 16A shows the base CTA particles at 500 times magnification, while FIG. 16B zooms in on one of the base CTA particles and shows that particle at 2,000 times magnification. As perhaps best illustrated in FIG. 16B, each base CTA particle is typically formed of a large number of small, agglomerated CTA subparticles, thereby giving the base CTA particle a relatively high surface area, high porosity, low density, and good dissolvability. The base CTA particles typically have a mean particle size in the range of from about 20 to about 150 microns, more preferably in the range of from about 30 to about 120 microns, and most preferably in the range of from 40 to 90 microns. The CTA subparticles typically have a mean particle size in the range of from about 0.5 to about 30 microns, more preferably from about 1 to about 15 microns, and most preferably in the range of from 2 to 5 microns. The relatively high surface area of the base CTA particles illustrated in FIGS. 16A and 16B, can be quantified using a Braunauer-Emmett-Teller (BET) surface area measurement method. Preferably, the base CTA particles have an average BET surface of at least about 0.6 meters squared per gram (m²/g). More preferably, the base CTA particles have an average BET surface area in the range of from about 0.8 to about 4 m²/g. Most preferably, the base CTA particles have an average BET surface area in the range of from 0.9 to 2 m²/g. The physical properties (e.g., particle size, BET surface area, porosity, and dissolvability) of the base CTA particles formed by optimized oxidation process of a preferred embodiment of the present invention permit purification of the CTA particles by more effective and/or economical methods, as described in further detail below with respect to FIG. 19.

The mean particle size values provided above were determined using polarized light microscopy and image analysis. The equipment employed in the particle size analysis included a Nikon E800 optical microscope with a 4x Plan Flour N.A. 0.13 objective, a Spot RT™ digital camera, and a personal computer running Image Pro Plus™ V4.5.0.19 image analysis software. The particle size analysis method included the following main steps: (1) dispersing the CTA powders in mineral oil; (2) preparing a microscope slide/cover slip of the dispersion; (3) examining the slide using polarized light microscopy (crossed polars condition—particles appear as bright objects on black background); (4) capturing different images for each sample preparation (field size=3×2.25 mm; pixel size=1.84 microns/pixel); (5) performing image analysis with Image Pro Plus™ software; (6) exporting the particle measures to a spreadsheet; and (7) performing statistical characterization in the spreadsheet. Step (5) of “performing image analysis with Image Pro Plus™ software” included the substeps of: (a) setting the image threshold to detect white particles on dark background; (b) creating a binary image; (c) running a single-pass open filter to filter out pixel noise; (d) measuring all particles in the image; and (e) reporting the mean diameter measured for each particle. The Image Pro Plus™ software defines mean diameter of individual particles as the number average length of diameters of a particle measured at 2 degree intervals and passing through the particle's centroid. Step 7 of “performing statistical characterization in the spreadsheet” comprises calculating the volume-weighted mean particle size as follows. The volume of each of the n particles in a sample is calculated as if it were spherical using pi/6*dᵢˆ3; multiplying the volume of each particle times its diameter to find pi/6*dᵢˆ4; summing for all particles in the sample of the values of pi/6*dᵢˆ4; summing the volumes of all particles in the sample; and calculating the volume-weighted particle diameter as sum for all n particles in the sample of (pi/6*dᵢˆ4) divided by sum for all n particles in the sample of (pi/6*dᵢˆ3). As used herein, “mean particle size” refers to the volume-weighted mean particle size determined according to the above-described test method; and it is also referred to as D(4,3).

$$D ( 4 , 3 ) = \frac{{\sum }_{i = 1}^{n} \frac{π}{6} {d}_{i}^{4}}{{\sum }_{i = 1}^{n} \frac{π}{6} {d}_{i}^{3}}$$

In addition, step 7 comprises finding the particle sizes for which various fractions of the total sample volume are smaller. For example, D(v,0.1) is the particle size for which 10 percent of the total sample volume is smaller and 90 percent is larger; D(v,0.5) is the particle size for which one-half of the sample volume is larger and one-half is smaller; D(v,0.9) is the particle size for which 90 percent of the total sample volume is smaller; and so on. In addition, step 7 comprises calculating the value of D(v,0.9) minus D(v,0.1), which is herein defined as the “particle size spread”; and step 7 comprises calculating the value of the particle size spread divided by D(4,3), which is herein defined as the “particle size relative spread.”

Furthermore, it is preferable that the D(v,0.1) of the CTA particles as measured above be in the range from about 5 to about 65 microns, more preferably in the range from about 15 to about 55 microns and most preferably in the range from 25 to 45 microns. It is preferable that the D(v,0.5) of the CTA particles as measured above be in the range from about 10 to about 90 microns, more preferably in the range from about 20 to about 80 microns, and most preferably in the range from 30 to 70 microns. It is preferable that the D(v,0.9) of the CTA particles as measured above be in the range from about 30 to about 150 microns, more preferably in the range from about 40 to about 130 microns, and most preferably in the range from 50 to 110 microns. It is preferable that the particle size relative spread be in the range from about 0.5 to about 2.0, more preferably in the range from about 0.6 to about 1.5, and most preferably in the range from 0.7 to 1.3.

The BET surface area values provided above were measured on a Micromeritics ASAP2000 (available from Micromeritics Instrument Corporation of Norcross, Ga.). In the first step of the measurement process, a 2 to 4 gram of sample of the particles was weighed and dried under vacuum at 50° C. The sample was then placed on the analysis gas manifold and cooled to 77° K. A nitrogen adsorption isotherm was measured at a minimum of 5 equilibrium pressures by exposing the sample to known volumes of nitrogen gas and measuring the pressure decline. The equilibrium pressures were appropriately in the range of P/P₀=0.01−0.20, where P is equilibrium pressure and P₀ is vapor pressure of liquid nitrogen at 77° K. The resulting isotherm was then plotted according to the following BET equation:

$$\frac{P}{{V}_{a} ( {P}_{o} - P )} = \frac{1}{{V}_{m} C} + \frac{C - 1}{{V}_{m} C} ( \frac{P}{{P}_{o}} )$$

where Vₐ is volume of gas adsorbed by sample at P, Vₘ is volume of gas required to cover the entire surface of the sample with a monolayer of gas, and C is a constant. From this plot, Vₘ and C were determined. Vₘ was then converted to a surface area using the cross sectional area of nitrogen at 77° K by:

$$A = σ \frac{{V}_{m}}{RT}$$

where σ is cross sectional area of nitrogen at 77° K, T is 77° K, and R is the gas constant.

As alluded to above, CTA formed in accordance with one embodiment of the present invention exhibits superior dissolution properties verses conventional CTA made by other processes. This enhanced dissolution rate allows the inventive CTA to be purified by more efficient and/or more effective purification processes. The following description addresses the manner in which the rate of dissolution of CTA can quantified.

The rate of dissolution of a known amount of solids into a known amount of solvent in an agitated mixture can be measured by various protocols. As used herein, a measurement method called the “timed dissolution test” is defined as follows. An ambient pressure of about 0.1 megapascal is used throughout the timed dissolution test. The ambient temperature used throughout the timed dissolution test is about 22° C. Furthermore, the solids, solvent and all dissolution apparatus are fully equilibrated thermally at this temperature before beginning testing, and there is no appreciable heating or cooling of the beaker or its contents during the dissolution time period. A solvent portion of fresh, HPLC analytical grade of tetrahydrofuran (>99.9 percent purity), hereafter THF, measuring 250 grams is placed into a cleaned KIMAX tall form 400 milliliter glass beaker (Kimble® part number 14020, Kimble/Kontes, Vineland, N.J.), which is uninsulated, smooth-sided, and generally cylindrical in form. A Teflon-coated magnetic stirring bar (VWR part number 58948-230, about 1-inch long with ⅜-inch diameter, octagonal cross section, VWR International, West Chester, Pa. 19380) is placed in the beaker, where it naturally settles to the bottom. The sample is stirred using a Variomag® multipoint 15 magnetic stirrer (H&P Labortechnik AG, Oberschleissheim, Germany) magnetic stirrer at a setting of 800 revolutions per minute. This stirring begins no more than 5 minutes before the addition of solids and continues steadily for at least 30 minutes after adding the solids. A solid sample of crude or purified TPA particulates amounting to 250 milligrams is weighed into a non-sticking sample weighing pan. At a starting time designated as t=0, the weighed solids are poured all at once into the stirred THF, and a timer is started simultaneously. Properly done, the THF very rapidly wets the solids and forms a dilute, well-agitated slurry within 5 seconds. Subsequently, samples of this mixture are obtained at the following times, measured in minutes from t=0: 0.08, 0.25, 0.50, 0.75, 1.00, 1.50, 2.00, 2.50, 3.00, 4.00, 5.00, 6.00, 8.00, 10.00, 15.00, and 30.00. Each small sample is withdrawn from the dilute, well-agitated mixture using a new, disposable syringe (Becton, Dickinson and Co, 5 milliliter, REF 30163, Franklin Lakes, N.J. 07417). Immediately upon withdrawal from the beaker, approximately 2 milliliters of clear liquid sample is rapidly discharged through a new, unused syringe filter (25 mm diameter, 0.45 micron, Gelman GHP Acrodisc GF®, Pall Corporation, East Hills, N.Y. 11548) into a new, labeled glass sample vial. The duration of each syringe filling, filter placement, and discharging into a sample vial is correctly less than about 5 seconds, and this interval is appropriately started and ended within about 3 seconds either side of each target sampling time. Within about five minutes of each filling, the sample vials are capped shut and maintained at approximately constant temperature until performing the following chemical analysis. After the final sample is taken at a time of 30 minutes past t=0, all sixteen samples are analyzed for the amount of dissolved TPA using a HPLC-DAD method generally as described elsewhere within this disclosure. However, in the present test, the calibration standards and the results reported are both based upon milligrams of dissolved TPA per gram of THF solvent (hereafter “ppm in THF”). For example, if all of the 250 milligrams of solids were very pure TPA and if this entire amount fully dissolved in the 250 grams of THF solvent before a particular sample were taken, the correctly measured concentration would be about 1,000 ppm in THF.

When CTA according to the present invention is subjected to the timed dissolution test described above, it is preferred that a sample taken at one minute past t=0 dissolves to a concentration of at least about 500 ppm in THF, more preferably to at least 600 ppm in THF. For a sample taken at two minutes past t=0, it is preferred that CTA according to the current invention will dissolve to a concentration of at least about 700 ppm in THF, more preferably to at least 750 ppm in THF. For a sample taken at four minutes past t=0, it is preferred that CTA according to the current invention will dissolve to a concentration of at least about 840 ppm in THF, more preferably to at least 880 ppm in THF.

The inventors have found that a relatively simple negative exponential growth model is useful to describe the time dependence of the entire data set from a complete timed dissolution test, notwithstanding the complexity of the particulate samples and of the dissolution process. The form of the equation, hereinafter the “timed dissolution model,” is as follows:
S=A+B*(1−exp(−C*t)), where
t=time in units of minutes;
S=solubility, in units of ppm in THF, at time t;
exp=exponential function in the base of the natural logarithm of 2;
A, B=regressed constants in units of ppm in THF, where A relates mostly to the rapid dissolution of the smaller particles at very short times, and where the sum of A+B relates mostly to the total amount of dissolution near the end of the specified testing period; and
C=a regressed time constant in units of reciprocal minutes.

The regressed constants are adjusted to minimize the sum of the squares of the errors between the actual data points and the corresponding model values, which method is commonly called a “least squares” fit. A preferred software package for executing this data regression is JMP Release 5.1.2 (SAS Institute Inc., JMP Software, SAS Campus Drive, Cary, N.C. 27513).

When CTA according to the present invention is tested with the timed dissolution test and fitted to the timed dissolution model described above, it is preferred for the CTA to have a time constant “C” greater than about 0.5 reciprocal minutes, more preferably greater than about 0.6 reciprocal minutes, and most preferably greater than 0.7 reciprocal minutes.

FIGS. 17A and 17B illustrate a conventional CTA particle made by a conventional high-temperature oxidation process in a continuous stirred tank reactor (CSTR). FIG. 17A shows the conventional CTA particle at 500 times magnification, while FIG. 17B zooms in and shows the CTA particle at 2,000 times magnification. A visual comparison of the inventive CTA particles illustrated in FIGS. 16A and 16B and the conventional CTA particle illustrated in FIGS. 17A and 17B shows that the conventional CTA particle has a higher density, lower surface area, lower porosity, and larger particle size than the inventive CTA particles. In fact, the conventional CTA represented in FIGS. 17A and 17B has a mean particle size of about 205 microns and a BET surface area of about 0.57 m²/g.

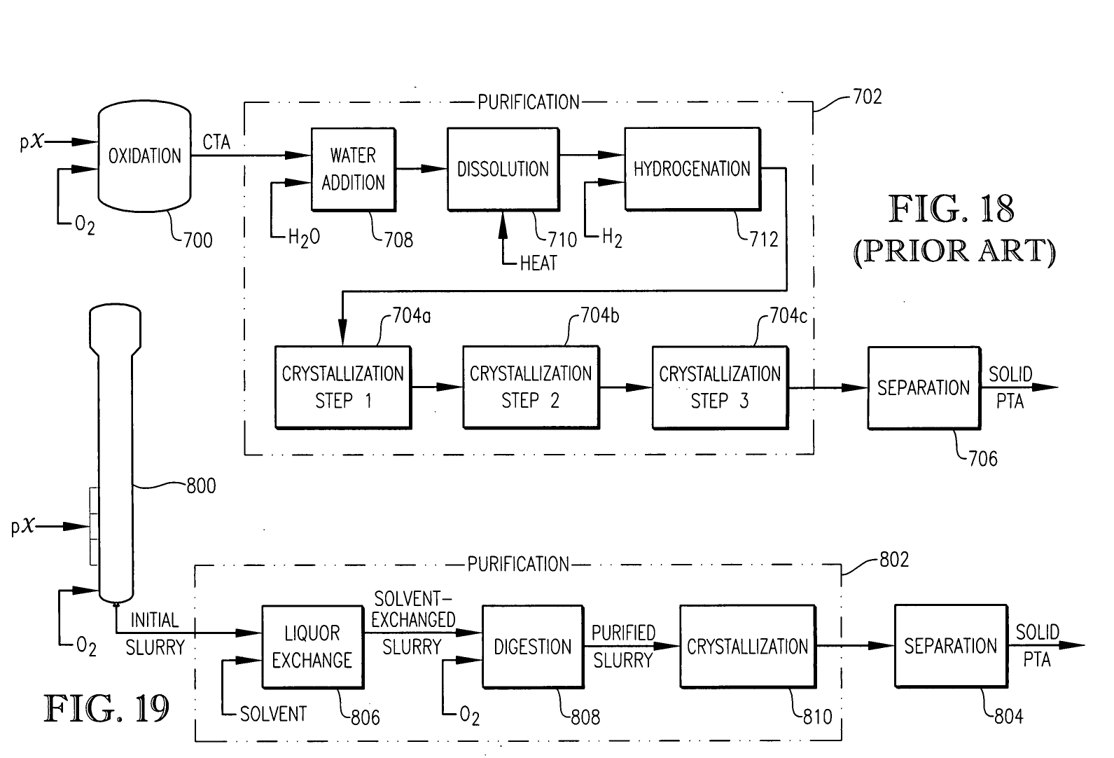

FIG. 18 illustrates a conventional process for making purified terephthalic acid (PTA). In the conventional PTA process, para-xylene is partially oxidized in a mechanically agitated high temperature oxidation reactor 700. A slurry comprising CTA is withdrawn from reactor 700 and then purified in a purification system 702. The PTA product of purification system 702 is introduced into a separation system 706 for separation and drying of the PTA particles. Purification system 702 represents a large portion of the costs associated with producing PTA particles by conventional methods. Purification system 702 generally includes a water addition/exchange system 708, a dissolution system 710, a hydrogenation system 712, and three separate crystallization vessels 704a,b,c. In water addition/exchange system 708, a substantial portion of the mother liquor is displaced with water. After water addition, the water/CTA slurry is introduced into the dissolution system 710 where the water/CTA mixture is heated until the CTA particles fully dissolve in the water. After CTA dissolution, the CTA-in-water solution is subjected to hydrogenation in hydrogenation system 712. The hydrogenated effluent from hydrogenation system 712 is then subjected to three crystallization steps in crystallization vessels 704a,b,c, followed by PTA separation in separation system 706.

FIG. 19 illustrates an improved process for producing PTA employing a bubble column oxidation reactor 800 configured in accordance with an embodiment of the present invention. An initial slurry comprising solid CTA particles and a liquid mother liquor is withdrawn from reactor 800. Typically, the initial slurry may contain in the range of from about 10 to about 50 weight percent solid CTA particles, with the balance being liquid mother liquor. The solid CTA particles present in the initial slurry typically contain at least about 400 ppmw of 4-carboxybenzaldehyde (4-CBA), more typically at least about 800 ppmw of 4-CBA, and most typically in the range of from 1,000 to 15,000 ppmw of 4-CBA. The initial slurry withdrawn from reactor 800 is introduced into a purification system 802 to reduce the concentration of 4-CBA and other impurities present in the CTA. A purer/purified slurry is produced from purification system 802 and is subjected to separation and drying in a separation system 804 to thereby produce purer solid terephthalic acid particles comprising less than about 400 ppmw of 4-CBA, more preferably less than about 250 ppmw of 4-CBA, and most preferably in the range of from 10 to 200 ppmw of 4-CBA.

Purification system 802 of the PTA production system illustrated in FIG. 19 provides a number of advantages over purification system 802 of the prior art system illustrated in FIG. 18. Preferably, purification system 802 generally includes a liquor exchange system 806, a digester 808, and a single crystallizer 810. In liquor exchange system 806, at least about 50 weight percent of the mother liquor present in the initial slurry is replaced with a fresh replacement solvent to thereby provide a solvent-exchanged slurry comprising CTA particles and the replacement solvent. The solvent-exchanged slurry exiting liquor exchange system 806 is introduced into digester (or secondary oxidation reactor) 808. In digester 808, a secondary oxidation reaction is preformed at slightly higher temperatures than were used in the initial/primary oxidation reaction carried out in bubble column reactor 800. As discussed above, the high surface area, small particle size, and low density of the CTA particles produced in reactor 800 cause certain impurities trapped in the CTA particles to become available for oxidation in digester 808 without requiring complete dissolution of the CTA particles in digester 808. Thus, the temperature in digester 808 can be lower than many similar prior art processes. The secondary oxidation carried out in digester 808 preferably reduces the concentration of 4-CBA in the CTA by at least 200 ppmw, more preferably at least about 400 ppmw, and most preferably in the range of from 600 to 6,000 ppmw. Preferably, the secondary oxidation temperature in digester 808 is at least about 10° C. higher than the primary oxidation temperature in bubble column reactor 800, more preferably about 20 to about 80° C. higher than the primary oxidation temperature in reactor 800, and most preferably 30 to 50° C. higher than the primary oxidation temperature in reactor 800. The secondary oxidation temperature is preferably in the range of from about 160 to about 240° C., more preferably in the range of from about 180 to about 220° C. and most preferably in the range of from 190 to 210° C. The purified product from digester 808 requires only a single crystallization step in crystallizer 810 prior to separation in separation system 804. Suitable secondary oxidation/digestion techniques are discussed in further detail in U.S. Pat. App. Pub. No. 2005/0065373, the entire disclosure of which is expressly incorporated herein by reference.

Terephthalic acid (e.g., PTA) produced by the system illustrated in FIG. 19 is preferably formed of PTA particles having a mean particle size of at least about 40 microns, more preferably in the range of from about 50 to about 2,000 microns, and most preferably in the range of from 60 to 200 microns. The PTA particles preferably have an average BET surface area less than about 0.25 m²/g, more preferably in the range of from about 0.005 to about 0.2 m²/g, and most preferably in the range of from 0.01 to 0.18 m²/g. PTA produced by the system illustrated in FIG. 19 is suitable for use as a feedstock in the making of PET. Typically, PET is made via esterification of terephthalic with ethylene glycol, followed by polycondensation. Preferably, terephthalic acid produced by an embodiment of the present invention is employed as a feed to the pipe reactor PET process described in U.S. Pat. No. 6,861,494, filed Dec. 7, 2001, the entire disclosure of which is incorporated herein by reference.

Oxidation bubble column reactors, such as the ones described above with reference to FIGS. 1-15, operate with flow fields that are highly chaotic and complex in a time-variant manner. This bubble column flow regime, which results at moderate to high superficial gas rates, is often called the churn-turbulent regime. Because the flow fields of a churn-turbulent bubble column are quite stochastic and because some oxidation reactions are rapid relative to the overall end-to-end mixing times of a bubble column, it is useful to model computationally certain aspects of the oxidation bubble column reactors using computational fluid dynamics (CFD) methods. For example, it is useful to use CFD to model the time-dependent and position-dependent aeration patterns, the time-dependent and position-dependent dispersion of the incoming feed stream of oxidizable compound, and/or the time-dependent and position-dependent concentration of dissolved oxygen in various parts of the reaction medium.

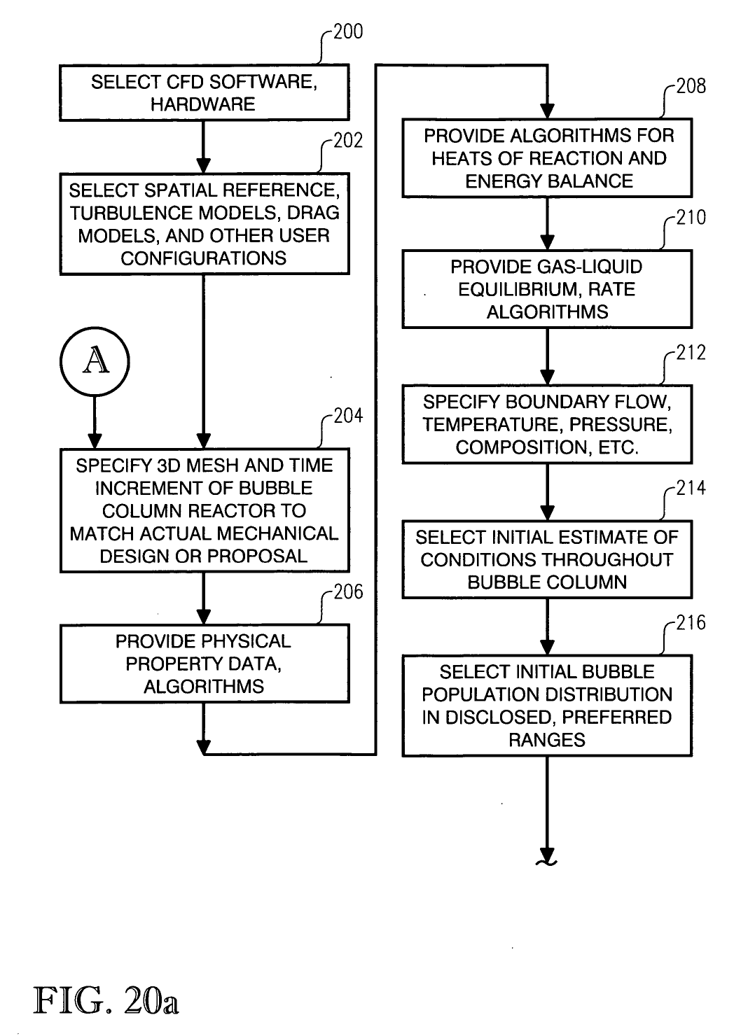

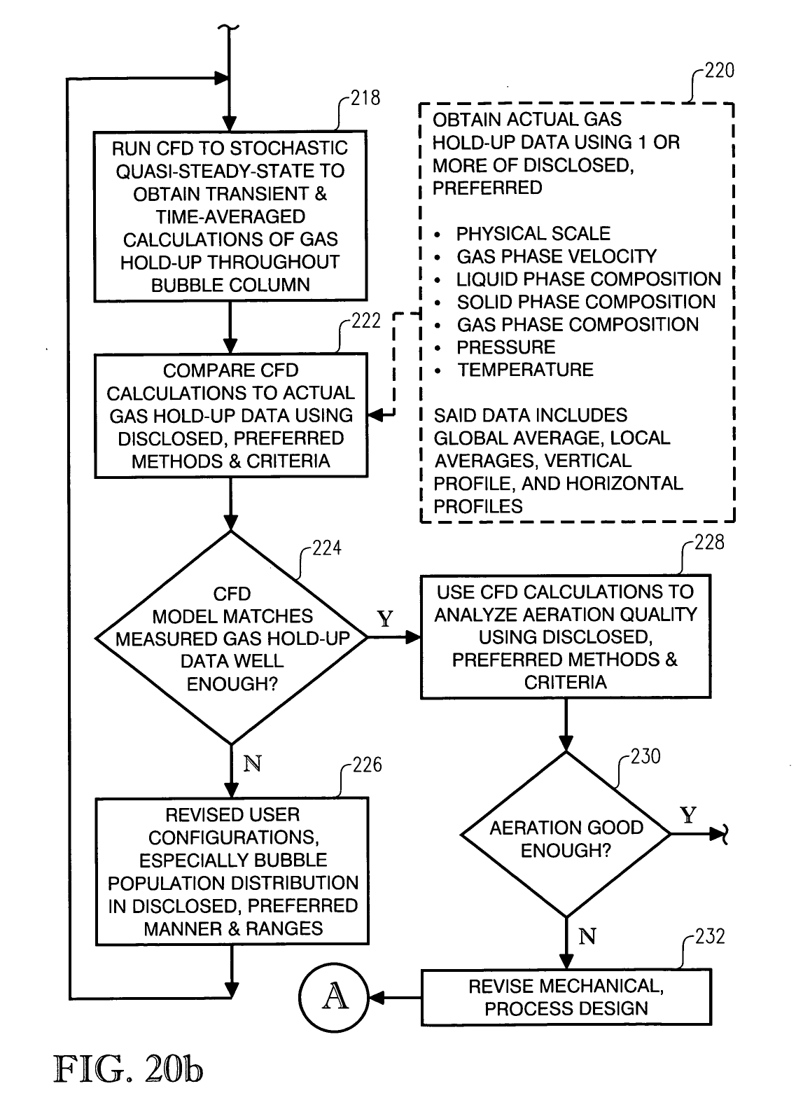

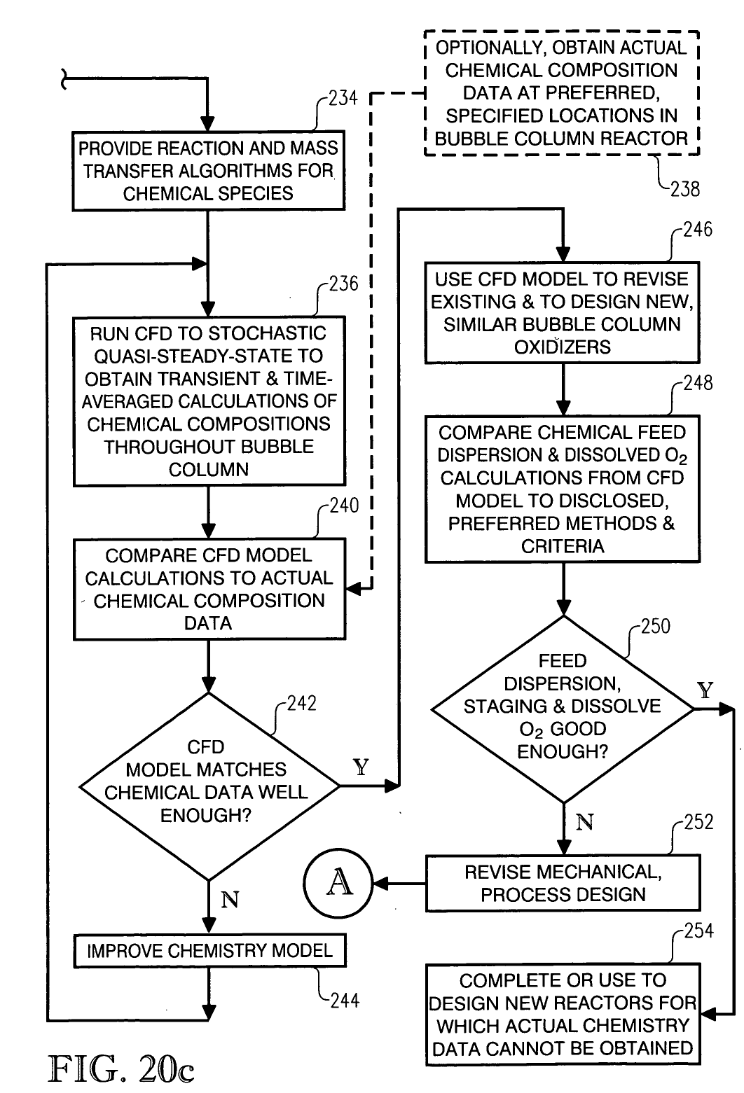

The computer modeling methods of the present invention will now be described, with reference to the flow diagrams illustrated in FIGS. 20 and 21. The modeling methods described below are preferably used to model an oxidation reactor configured and operated in accordance with the description provided above with reference to FIGS. 1-19

In step 200 (FIG. 20a) of the inventive method, appropriate CFD modeling software and hardware is selected. Various commercially available CFD modeling software packages can be employed in the present invention. Suitable examples include Fluent (Fluent, Inc., 10 Cavendish Court, Centerra Park Lebanon, N.H. 03766) and CFX version 5.7 (ANSYS, Inc., 275 Technology Drive, Canonsburg, Pa. 15317). The hardware on which the CFD software is run can be selected from a number of commercially available computer hardware systems. For example, the CFD software can be installed and executed on 8-16 personal computers (PCs) running in parallel.

In step 202, appropriate spatial reference, turbulence models, drag models, and other user configurations for the CFD model are selected. The CFD model of the instant invention is preferably three dimensional (3D) Eulerian-Eulerian. This type of model is computationally intensive, but two dimensional Eulerian-Eulerian models may lack fidelity on important stochastic features of the churn-turbulent regime. On the other hand, Lagrangian computational models of this system are even more computationally intensive and are currently largely impractical. Preferably, both the liquid phase and the gas phase models are of the Reynolds-Averaged Navier-Stokes (RANS) family in which small scale fluctuations are added to the mean flow by superposition and then time-averaged. Transient flow features are still captured, but only those of a larger scale. Several turbulence models are provided as standard within typical commercial CFD packages, allowing for user selection. Various turbulence models may be usefully employed for modeling bubble column oxidation reactors. For the liquid phase, preferred turbulence models include k-epsilon and k-omega turbulence models, which are linear two-equation models using the eddy-viscosity hypothesis. More preferably, the turbulence model employed for the liquid phase is a Shear Stress Transport (SST) variant of the k-omega model (turbulence-kinetic-energy—turbulence-frequency). Preferably, the turbulence model employed for the gas phase is a zero-equation model, which means the turbulence effects of the gas phase are a function of the turbulence effects of the liquid phase. Many drag models may be employed to estimate the force interaction between the liquid (or slurry pseudo-liquid, see below) and gas phases. A preferred method utilizes the Grace Drag Law, which takes into account the effects of bubble shape, bubble size, and bubble swarming in computing the drag force between the gas and the liquid. All of these features exist as user selected options in the commercial CFD software packages disclosed above.

In step 204, the 3D mesh and time increment of the bubble column model are specified to match the actual or proposed mechanical design. The 3D computational meshes employed in the CFD computational models of the instant invention preferably utilize upwards of about 1,000 computational cells, more preferably between about 10,000 and about 3,000,000 computational cells, and most preferably between 50,000 and 1,000,000 computational cells. Computational time increases super-linearly with the number of computational cells, but models with too few computational cells lack fidelity for oxidation reactions conducted with rapid reaction rates and for the high levels of stochastic flow chaos occurring in bubble columns operating well into the churn-turbulent regime. The computational cell shapes (e.g. tetrahedral, prismatic, and so on), aspect ratio, growth rate, and linear dimensions are adjusted throughout the column to balance computational intensity and model fidelity. Owing to the relatively complex physical geometry near the air inlet(s) and in the bottom vessel head and owing to the importance of spatial resolution near the feed inlet(s) for the oxidizable compound, it is preferred for the cell counts per unit volume to be relatively higher in these regions.

The time increment of computation is selected to give numerical stability and resolution with the given meshing and other modeling assumptions. Also, process conditions within the column have an effect on what is an appropriate time increment. For example, the time increment required may be shorter when the CFD model is subjected to large transients (e.g., during a model start-up condition, after model feed rate changes, and/or after a model pressure change). Preferably, model time increments of less than about one second are used, more preferably less than about 0.5 seconds, and most preferably less than 0.1 seconds, even after the CFD model has reached stochastic quasi-steady-state.

In step 206, physical property data and algorithms are provided to the CFD model. Commercially-available CFD software requires user input of various pertinent physical properties (e.g., density, viscosity, and surface tension) of the gas, liquid, and solid phases. The pertinent physical properties of the gas portion of the reaction medium can be measured for relevant process conditions (e.g. temperature, pressure, and composition). Optionally, there are many methods known to calculate with useful accuracy these physical property inputs. A preferred method of estimating gas-phase physical properties is using Aspen Plus® version 12.1 (Aspen Technology, Inc., Ten Canal Park, Cambridge, Mass. 02141). For the particular case of an oxidation of para-xylene to terephthalic acid, the gas portion of the reaction medium typically comprises major quantities of acetic acid vapor, water vapor, oxygen, and nitrogen (unless pure molecular oxygen is fed without significant nitrogen), along with lesser quantities of many additional minor components, including carbon monoxide, carbon dioxide, methyl acetate, and para-xylene. However, the four major species (three, if no nitrogen) are generally sufficient to approximate the aggregated density, viscosity, and other properties of the gas phase. Whether gas properties are measured or estimated, the inventors have discovered that many values of pertinent physical properties appropriate to the centroid of the reaction medium are usefully held constant for CFD calculations throughout said reaction medium. In bubble columns where gas density varies significantly due to changes in temperature and pressure from place to place within the reaction medium, it is preferred to use the Ideal Gas Law correlation, namely that gas density varies directly with the ratio of absolute pressure and inversely with the ratio of absolute temperature, applied to the values measured or estimated for the centroid.

If there is no solid phase in the bubble column, the pertinent physical properties of the liquid phase of the reaction medium are measured for relevant process conditions (e.g., temperature, pressure, composition, and shear rates). Optionally, there are many methods known to calculate with useful accuracy the pertinent physical properties of the liquid phase for input to CFD. A preferred method of estimating liquid-phase physical properties is using Aspen Plus® version 12.1 (Aspen Technology, Inc., Ten Canal Park, Cambridge, Mass. 02141). Whether liquid properties are measured or estimated, the inventors have discovered that values of the liquid properties pertinent to the centroid of the reaction medium are usefully held constant for CFD calculations throughout the reaction medium.

If the bubble column comprises solids, the slurry portion of the reaction medium can be modeled as separate liquid and solid phases. This is particularly useful when the concentration of solids varies significantly according to position within the bubble column. For example, such variation may come by gravimetric settling, locally high precipitation or dissolution of solids, locally high evaporation of solvent, and/or segregation of solids according to shear fields. However, if the distribution of solids is observed to be sufficiently uniform, it is computationally preferable that the slurry is modeled as a pseudo-single-phase using a pseudo-liquid with a pseudo-density, pseudo-viscosity, and so on for the appropriate mixture of major liquid components and for the appropriate fraction and characteristics of the solids. In a churn-turbulent bubble column carrying out oxidation of para-xylene to terephthalic acid, the solids distribution is often sufficiently uniform such that the slurry portion of this reaction medium is more preferably modeled as a single phase pseudo-liquid for the appropriate mixture of liquid components (e.g., acetic acid liquid, water liquid, and/or oxidizable compound liquid) and for the appropriate fraction and characteristics of the solids (e.g., terephthalic acid).

In step 208, algorithms for heats of reaction and energy balance are provided to the model. In bubble column reactors operated in accordance with an embodiment of the present invention, one or more chemical reactions are carried out, each with an associated heat of reaction. These heats of reaction may appreciably alter the temperature and/or physical properties of the material within the bubble column and/or portions thereof, perhaps even affecting whether the material contained is present in solid, liquid, or gaseous form. The reactions may be endothermic or exothermic, and they may be mostly uniform or greatly variant with respect to time and position. For bubble column oxidation reactors, the heats of reaction are typically substantial exothermic amounts, and it is preferable in the present invention that the model include algorithms for calculating the heats of reaction. Furthermore, it is preferred that models of the present invention also include algorithms for maintaining the net energy balance on the overall bubble column reactor and/or local positions thereof. These energy balance algorithms may include models for any or all of the various means used in actual physical reacting systems, including but not limited to algorithms for heat exchange surfaces and algorithms for the enthalpy of material flowing into and out of the overall reactor and/or local portions thereof. The commercial CFD software disclosed above is amenable to accepting these algorithms for the heats of reaction and the energy balance.

In step 210, algorithms for gas-liquid equilibrium and inter-phase mass transfer rates are provided. In oxidation bubble columns, it is common for a large portion of the heat of reaction to be removed from the reaction medium by evaporating a significant amount of the liquid phase. In fact, a substantial amount of vaporization from the liquid phase typically occurs wherever the oxidant feed stream is first introduced into the reaction medium. Thus, the gas phase of the reaction medium at virtually every position and time typically comprises considerable evaporated solvent, and even evaporated oxidizable compound, in addition to the initial components of the oxidant feed stream. In fact, the mass and/or molar flow rate of evaporated solvent out of the top of the reaction medium may often approach or even greatly exceed the inlet flow rate of the oxidant feed stream.

In oxidation bubble columns, the amount of the evaporated solvent extant in the gas phase at any one position in the reaction medium is a very complicated and dynamic balance. As the oxidant stream travels up the reactor, the static pressure is reduced since the amount of liquid head is reduced; this reduced pressure often induces appreciably more evaporation of the liquid phase. As a countervailing factor, molecular oxygen is consumed as the oxidant stream travels up the reactor; this consumption of oxygen reduces the amount of supercritical gas species in the gas phase, typically significantly reducing the equilibrium amount of evaporated liquid phase for a given pressure and temperature. In addition, there may be temperature gradients within the reaction medium, and these affect the thermodynamic equilibrium between the liquid and gas phases. In further addition, equilibrium between the gas and liquid phases, although rapidly obtained, is not instantly obtained. For bubble columns operating in the churn-turbulent flow regime, there is a considerable stochastic variation of flow patterns interacting with all of the other factors mentioned above. Thus, the amount of evaporated liquid in the gas phase varies within the bubble column reactor according to numerous factors involving both space (position) and time.

It is preferred for CFD models of the present invention to account for the amount of evaporated liquid phase occurring in various parts of the bubble column. Failure to consider the effects of the evaporated liquid phase can lead to large errors in modeling. It is more preferable that calculations of the amount of evaporated liquid be based on thermodynamic calculations comprising the heat of reaction; and/or the sensible heat of feed streams of solvent, oxidizable compound, and oxidant; and/or the heat capacity of the liquid, or slurry, and gas phases of the reaction medium; and/or the heat of vaporization and/or condensation of the liquid and/or gas phase; and/or the vapor-liquid thermodynamic equilibrium relations as a function of pressure, temperature, and composition; and/or the local pressure at positions within the reaction medium; and/or the local temperature at positions within the reaction medium. It is most preferable that these thermodynamic estimations are augmented by estimations of the rate of mass transport between the liquid and gas phases so that the amount of gas phase can be appropriately estimated at various positions within the bubble column. Estimation of these mass transfer rates also has utility in calculating the amount of dissolved molecular oxygen at various positions and times throughout the reaction medium.

In step 212, the relevant process boundary conditions relating to entering and exiting flow rates, compositions, pressures, and temperatures are provided to the CFD model.

In step 214, an initial estimate of the conditions throughout the bubble column is provided to the CFD model configuration. The appropriateness of this initial estimation of pressure, temperature, and composition within the various cells of the computational mesh may greatly affect the speed at which the CFD software converges to the quasi-steady-state model. Often, it is preferable that the initial estimation be somewhat close to the anticipated quasi-steady-state conditions.

In step 216, initial estimates of bubble sizes are provided for different parts of the reaction medium. In an actual bubble column operating in the churn-turbulent regime, seemingly limitless numbers of different sizes of individual bubbles and bubble swarms exist. There is constant coalescence and break-up of bubbles and swarms, always in a highly chaotic way. Notwithstanding the short term chaos, the size and number of bubbles are known to vary according to position within the bubble column when considered in a time-averaged sense. Though ultimate computational fidelity presently remains beyond reach, useful fidelity can be had by approximating that a quasi-stable bubble size population exists in a time-averaged sense. One, two, or more different average bubble sizes are typically used. Often the quasi-stable bubble size population will vary according to position in the column.

In step 218 (FIG. 20b), CFD calculations are commenced and allowed to proceed to a stochastic quasi-steady-state (i.e., a dynamic and chaotic quasi-equilibrium).

The inventors have discovered that for certain oxidation bubble columns it is not yet possible with existing CFD codes to input physical properties, turbulence models, drag models, off-the-shelf bubble size models, thermodynamic models for temperature, thermodynamic models for vapor-liquid equilibrium, and/or models of vapor-liquid mass transfer rates, and thereby obtain a priori fidelity versus actual operating oxidation bubble columns. The inventors have also discovered that for certain disclosed oxidation bubble columns it is necessary to determine actual gas hold-up information for conditions sufficiently closely approximating the intended modeling conditions. This actual data for gas hold-up can then be used to tune various parameters in the CFD model to obtain calculational fidelity. Specifically, for oxidation bubble columns operating with the elevated temperatures, vapor pressures, gas phase densities, high superficial gas velocities, physical size, space-time-reaction rates, chemical gradients, thermal gradients, and solids loadings, the inventors have discovered that gas hold-up according to a CFD model may change dramatically within various parts of the bubble column, from far too low a bubble hold-up, to a credible churn-turbulent regime with appropriate bubble hold-up, all the way to an erroneously foamy condition, owing to seemingly small changes in some user assigned variables (e.g. drag model assumptions, bubble population assumptions, and surface tension estimations and variations). Unless fidelity is approached between actual/measured data for gas hold-up and the modeled gas hold-up of the CFD model, the further details of the CFD model for the flow fields and mixing within the bubble column reactor are apt to be in great error. These flow errors will propagate further when chemistry models of the oxidation reactions are added.

In step 220, actual/measured gas hold-up data is obtained from an operating actual bubble column reactor. Preferably, the actual bubble column reactor is configured and operated in accordance with the description provided above with reference to FIGS. 1-19. The actual/measured data for gas hold-up is preferably obtained from an operating bubble column reactor containing a reaction medium having a maximum width (W) in excess of about 0.2 meters, more preferably between about 1 and about 15 meters, and most preferably between 2 and 10 meters. Preferably, the measured data for gas hold-up is obtained with the maximum height (H) of the reaction medium in excess of about 0.5 meters, more preferably between about 2 and about 90 meters, and most preferably between 5 and 50 meters. Preferably, the measured data for gas hold-up is obtained with the H:W ratio of the reaction medium being in the range of from about 2:1 to about 30:1, still more preferably in the range of from about 3:1 to about 20:1, and most preferably in the range of from 4:1 to 12:1.

Preferably, the measured data for gas hold-up is obtained from an operating bubble column reactor containing a reaction medium having a superficial gas phase velocity in excess of about 0.2 meters per second, more preferably between about 0.4 and 6 meters per second, still more preferably between about 0.6 and 3 meters per second, and most preferably between 0.8 and 2 meters per second.

Preferably, the measured data for gas hold-up is obtained from an operating bubble column reactor containing a reaction medium having a liquid phase comprising carboxylic acids. More preferably, the measured data for gas hold-up is obtained from a reaction medium having a liquid phase comprising water and carboxylic acids. Most preferably, the measured data for gas hold-up is obtained from a reaction medium having a liquid phase comprising water and at least 50 weight percent acetic acid.

Preferably, the measured data for gas hold-up is obtained from an operating bubble column reactor containing a reaction medium having a gas phase with an average molecular weight exceeding about 30 grams per gram-mole. More preferably, the measured data for gas hold-up is obtained from a reaction medium having a gas phase with an average molecular weight exceeding about 35 grams per gram-mole and comprising water vapor. Most preferably, the measured data for gas hold-up is obtained from a reaction medium having a gas phase with an average molecular weight exceeding 40 grams per gram-mole and comprising water vapor and acetic acid vapor.

If precipitated solids are present, it is preferable that the measured data for gas hold-up is obtained from an operating bubble column reactor containing a reaction medium having a solids content of above about 4 weight percent of total slurry weight, more preferably between about 8 and about 45 weight percent of total slurry weight, and most preferably between 15 and 35 weight percent of total slurry weight. Preferably, the measured data for gas hold-up is obtained from an operating bubble column reactor containing a reaction medium having a solids content in the slurry within about 15 weight percent of the intended modeling condition, more preferably within about 10 weight percent of the intended modeling condition, and most preferably within 3 weight percent of the intended modeling condition. For example, if the model target is 31 weight percent solids in the slurry, then the most preferred range for obtaining measured data for gas hold-up is 28 to 34 weight percent solids.

If precipitated solids are present, it is preferable that the measured data for gas hold-up is obtained from an operating bubble column reactor containing a reaction medium having a median solid particle size between about 5 and about 200 microns, more preferably between about 10 and about 150 microns, still more preferably between about 20 and about 100 microns, and most preferably the measured data for gas hold-up is obtained from a reaction medium, having a median particle size that matches the median particle size of the model.

Preferably, the measured data for gas hold-up is obtained from an operating bubble column reactor containing a reaction medium having a pressure above about 0.05 megapascal gauge, more preferably between about 0.2 and about 3 megapascal gauge, and most preferably between 0.3 and 1.5 megapascal gauge. Preferably, the measured data for gas hold-up is obtained from an operating bubble column reactor containing a reaction medium having an absolute pressure within about 0.7 megapascal gauge of the intended modeling condition, more preferably within about 0.5 megapascal gauge of the intended modeling condition, still more preferably within about 0.3 megapascal gauge of the intended operating condition, and most preferably within 0.1 megapascal gauge of the intended modeling condition.

Preferably, the measured data for gas hold-up is obtained from an operating bubble column reactor containing a reaction medium having an absolute temperature above about 50° C., more preferably between about 50 and about 250° C., still more preferably between about 100 and about 22020 C., and most preferably within 10° C. of the intended modeling condition.

Preferably, measured data for gas hold-up is obtained from an operating bubble column reactor containing a reaction medium having an average gas hold-up measured over at least about 60 percent, more preferably at least about 80 percent, and most preferably approximately all of the height of the reaction medium. One convenient method to obtain this data is using differential pressure measurement from the base of the reactor to a location in the gas headspace above the reaction medium along with measurement of the position of the top interface of the reaction medium. One convenient method to locate the top interface of the reaction medium is by using a gamma-radiation-emitting and detection method to locate the density change for the top interface of the reaction medium. By calculation, the observed differential pressure can be converted to a mass of reaction medium. By further calculation, the volume occupied by the aerated reaction medium is determined and compared with the volume that would be occupied by unaerated liquid, or slurry, at the appropriate pressure and temperature. The fraction of gas hold-up is thus determined. Other means of detecting the average gas hold-up are known and possible.

More preferably, measured data for gas hold-up of the operating bubble column is additionally obtained for one or more elevation spans wherein each span is significantly less than the total height of the reaction medium. One means to obtain this data is by measurement of differential pressure between two locations spaced vertically in the reaction medium by a known height difference. Another means to obtain this type of measured data for gas hold-up is by measurement of the mass of the reaction medium at a specific height, or range of height, using a gamma-radiation-emitting and detection method across a known path length of reaction medium. This method involves locating a gamma-radiation-emitting source near the reaction medium, conveniently on an external wall of the bubble column reactor vessel but possibly enveloped within the reaction medium, at a given elevation, locating a gamma-radiation detection and measurement device at about the same elevation and often diametrically across the vessel from the source, and obtaining a time-averaged determination of the amount of radiation transiting the path from the source though the reaction medium and reaching the detector. Then, the attenuation of the radiation signal is compared to the attenuation that would be detected were the radiation path occupied by unaerated liquid, or slurry, near the pressure and temperature of the reaction medium, and a volume fraction of gas hold-up is computed. Many such radiation emission/detection devices are available commercially from vendors such as Ohmart (Ohmart/VEGA, 4170 Rosslyn Drive, Cincinnati, Ohio 45209) and Ronan (Ronan Engineering Company, 8050 Production Drive, Florence, Ky., 41042, USA), among others. When using a gamma-radiation-emitting and detection method, it is preferred for a profile of radiation measurements to be obtained with an empty, idle bubble column vessel and then repeated for the same elevations and path lengths while operating with reaction medium. The empty, idle profile provides a more accurate correction for the presence of the mass of the reaction vessel, insulation, and so on in the radiation path than does estimation of these corrections from physical dimensions, materials, and radiation attenuation models.

Still more preferably, measured data for gas hold-up includes a vertical profile obtained by repeating the measurement of the gas hold-up of the reaction medium for at least two elevations vertically separated from lowest to highest by more than about 20 percent of the total height of the reaction medium, more preferably for at least three elevations vertically separated from lowest to highest by at least about 50 percent of the total height of the reaction medium, and most preferably for at least four elevations with vertical separation of at least 90 percent of the total height of the reaction medium.

Even more preferably, measured data for gas hold-up includes at least one horizontal, cross-sectional gas hold-up profile. Importantly, the time-averaged axial flows within the bubble column are known to correlate well with the time-averaged cross-sectional gas hold-up profiles. A preferred means of obtaining this type of measured data for gas hold-up is by using computed tomography (CT) scanning using gamma-radiation-emitting and detection methods. The method is somewhat similar to the vertical gas hold-up profile, except that a horizontal CT scan determines gas hold-up measurements across a number of different paths, both diameters and chords, at about the same elevation. A more preferred method of CT involves obtaining a fan-shaped pattern of gas hold-ups along chords at a given elevation. First, a gamma-radiation-emitting source is placed at one location on the side of the vessel and at least one detector, more preferably at least 4, and most preferably at least 8, is (are) located at various positions around the circumference of the vessel to detect the signal strength along different paths simultaneously. Then the gamma-radiation-emitting source is relocated to at least one different position, more preferably at least 4 different positions, and most preferably at least 8 different positions, at about the same elevation and the fan shaped pattern of detectors is repeated along the additional gas hold-up chords. Most preferably, measured data for gas hold-up comprises at least two horizontal, cross-sectional gas hold-up profiles obtained for two elevations separated by at least 30 percent of the total height of the reaction medium. The acquisition of data and reconstruction of the vertical and horizontal gas hold-up profiles using gamma-radiation-emitting and detection methods is available on a commercial, contractual basis from multiple contractors, including for example Tracerco (Houston, Tex.) and Quest TruTec (La Porte, Tex.)

In accordance with step 222, the preliminary CFD calculations are compared to the measured data for gas hold-up. In comparing the CFD model calculations to measured data for gas hold-up, it is preferable to recognize the stochastic nature of the system by time-averaging the CFD model calculations through times lasting at least about 10 seconds. More preferably, the CFD model calculations are time-averaged through times lasting at least about 100 seconds. Most preferably, the CFD model calculations are time-averaged through times lasting between 100 seconds and 1,000 seconds.

In comparing the CFD model calculations to measured data for gas hold-up, it is preferable that the time-averaged, volume-averaged modeled gas hold-up for the entire modeled reaction medium be within about 0.9 and 1.1 times the measured gas hold-up for the entire actual reaction medium. For example, if the time-averaged, volume-averaged measured gas hold-up for the entire reaction medium is 50 percent, it is preferable that the CFD model gas hold-up value is between 45 and 55 percent. It is more preferable that the time-averaged, volume-averaged modeled gas hold-up for the entire modeled reaction medium be within about 0.95 and 1.05 times the measured gas hold-up for the entire actual reaction medium. It is most preferable that the time-averaged, volume-averaged modeled gas hold-up for the entire modeled reaction medium be within 0.98 and 1.02 times the measured gas hold-up for the entire actual reaction medium.

In comparing the CFD model calculations to measured data for gas hold-up, it is preferable that the time-averaged, volume-averaged modeled gas hold-up for the one-quarter elevation, the mid-elevation, and the three-quarter elevation of the modeled reaction medium be within about 0.9 and 1.1 times the measured gas hold-up for the respective elevations of the actual reaction medium. For example, if the time-averaged, volume-averaged measured gas hold-up for the respective elevation is 50 percent, it is preferable that the CFD model gas hold-up value is between 45 and 55 percent for the same elevation. It is more preferable that the time-averaged, volume-averaged modeled gas hold-up for said elevations be within about 0.95 and 1.05 times the measured gas hold-up for the respective elevations. It is most preferable that the time-averaged, volume-averaged modeled gas hold-up for said elevations be within 0.98 and 1.02 times the measured gas hold-up for the respective elevations.

In comparing the CFD model calculations to measured data for gas hold-up, it is preferable that the time-averaged, volume-averaged horizontal profile of the modeled gas hold-up for the one-quarter elevation, the mid-elevation, and the three-quarter elevation of the modeled reaction medium match with the measured gas hold-up as follows. It is preferable that the modeled gas hold-up of the most central 9 percent of the cross-sectional area of the modeled reaction medium in the CFD model for each said elevation be within about 0.9 and 1.1 times the measured gas hold-up for said area for the respective elevation. For example, for each said height in a vertically cylindrical bubble column, if the reference time-averaged, volume-averaged measured gas hold-up for the cross-section of the column from the centroid of the area out to a radius of 0.3 D/2 is 60 percent, it is preferable that the modeled gas hold-up value for the same cross-sectional area is between 54 and 66 percent. It is more preferable that the modeled gas hold-up of the most central 9 percent of the cross-sectional area of the modeled reaction medium for each said height be within about 0.95 and 1.05 times the measured gas hold-up for said area. It is most preferable that the modeled gas hold-up of the most central 9 percent of the cross-sectional area of the modeled reaction medium for each said height be within 0.98 and 1.02 times the measured gas hold-up for said area.

In comparing the CFD model calculations to measured data for gas hold-up, it is preferable that the time-averaged, volume-averaged horizontal profile of the modeled gas hold-up for the one-quarter elevation, the mid-elevation, and the three-quarter elevation of the modeled reaction medium further match with the measured gas hold-up as follows. It is preferable that the modeled gas hold-up of the area simultaneously lying outside of the most central 64 percent of the cross-sectional area of the modeled reaction medium and inside of the most central 81 percent of the cross-sectional area of the modeled reaction medium for each said elevation be within about 0.9 and 1.1 times the measured gas hold-up for said area for the respective elevation. For example, for each said height in a vertically cylindrical bubble column, if the time-averaged, volume-averaged measured gas hold-up for the cross-section of the column lying in the annulus between a radius of 0.8 D/2 and a radius of 0.9 D/2, both measured from the centroid of the area, is 40 percent, then it is preferable that the modeled gas hold-up for the same cross-sectional area is between 36 and 44 percent. It is more preferable that the modeled gas hold-up of the area simultaneously lying outside of the most central 64 percent of the cross-sectional area of the modeled reaction medium and inside of the most central 81 percent of the cross-sectional area of the modeled reaction medium for each said elevation be within about 0.95 and 1.05 times the measured gas hold-up for the area for the respective elevation. It is most preferable that the modeled gas hold-up of the area simultaneously lying outside of the most central 64 percent of the cross-sectional area of the modeled reaction medium and inside of the most central 81 percent of the cross-sectional area of the modeled reaction medium for each said elevation be within 0.98 and 1.02 times the measured gas hold-up for the area for the respective elevation.

In step 224, the decision is made as to whether the CFD model matches the measured gas hold-up data well enough. If the various comparisons of the modeled and measured gas hold-up indicate acceptable agreement, then the vast additional output of the computational model is deemed useful for analysis and actions as described in disclosure further below. However, if there is insufficient agreement between the modeled and measured gas hold-up, then adjustment of the configuration of the CFD model is indicated before rerunning the model to obtain improved reconciliation with the measured gas hold-up data. If the CFD model data matches the measured gas hold-up data well enough, the inventive method proceeds to step 228, if not the method proceeds to step 226.

In step 226, fidelity of CFD calculations compared to the measured data for gas hold-up is obtained by adjusting the one or more user specified parameters within the CFD code, using successive model iterations as necessary. More preferably, this reconciliation of the CFD model with measured data is obtained by using at least two different bubble sizes within the CFD model. Still more preferably, the bubble populations are adjusted according to a vertical function in the reaction medium. Most preferably, the fractions of bubbles of various sizes are adjusted using from three to 16 different bubble sizes and using a vertical and horizontal function in the reaction medium. Preferably, the bubbles range in specified size upwards from about 0.001 meters in diameter. More preferably, the bubbles range in specified size from about 0.002 to about 0.3 meters in diameter. Most preferably, the bubbles range in specified size from 0.005 to 0.2 meters in diameter. The inventors have discovered that seemingly small changes in the specified fractions of various sizes of bubbles can cause the CFD model calculations to deviate dramatically from known, observed behavior for oxidation bubble columns. A realistic CFD model of flow fields obtained according to the present invention produces large scale fluctuations in the flow of bubble swarms and concomitant liquid surges that are generally consistent with the observed low frequency undulation in the operating actual bubble column reactor.

In accordance with step 228, once CFD model parameters have been adjusted to obtain fidelity with the measured data for gas hold-up, the output of the CFD model is used to evaluate the quality of aeration throughout the reaction medium. That is, the CFD calculations are inspected to ascertain whether mechanical modifications are appropriate to improve aeration. For example, various thresholds of gas hold-up (e.g., gas hold-up less than 0.1, gas hold-up less than 0.2, and so on) are used for discriminating which ones of 2,000 discrete horizontal slices of equal volume are likely to be poorly aerated, and mechanical and process modifications are considered to eliminate these poorly aerated regions.

In step 230, the decision is made as to whether the aeration is good enough. If aeration is good enough, the inventive method proceeds to step 234 (FIG. 20c). If the aeration is not good enough, the method proceeds to step 232, where the mechanical and/or process design is revised. After step 232, the method can return to step 204.

Once CFD model parameters, such as drag models and bubble size populations, have been adjusted to obtain fidelity with the measured data for gas hold-up, these CFD model parameters are useful to design completely new reactors with reaction medium at suitably similar ranges of various parameters described herein. This is important for oxidation bubble columns, because the flow patterns and mixing are critical to the chemistry of various competing, parallel and sequential reactions and because the flow patterns and mixing are driven by natural convection force balances that are highly dependent both on geometry and on scale.

In step 234, reaction and mass transfer algorithms for the chemical species are provided. To develop a computational model of chemical reactions within a bubble column oxidation reactor, obtaining fidelity between the CFD model and measured data for gas hold-up is merely a first step, providing the flow fields with appropriate stochastic and 3D fidelity. To consider the chemistry in greater detail, the computational modeling will also account for the reactive consumption, reactive creation, and inter-phase transport of one of more specific chemical species in addition to the convective and diffusive flows of the fluid dynamic model. Thus, it is useful to add computational models of varying complexity pertaining to one or more chemical species having chemical reactivity and/or chemical affinity for different phases (solid, liquid, gas). For example, in the oxidation of para-xylene to TPA, dispersion of para-xylene within the liquid phase is studied, or dispersion of para-xylene within both the liquid and gas phases is studied, or a reaction model involving creation and consumption of para-tolualdehyde, para-toluic acid, and 4-CBA is studied, or the concentrations of molecular oxygen in the gas phase and/or liquid phase are studied.

The inventors have discovered that usage of one or more reactive tracer species is particularly useful in regard to modeling oxidation bubble columns. The computational model components added to track various chemical species are herein referred to as “reactive” because models of the chemical reaction rates and equilibriums and/or phase partitioning rates and equilibriums are added into the model configuration as functions of temperature, pressure, composition, and so on of the reaction medium. The preferred commercial software packages are amenable in this respect, but the user must provide the appropriate functional form and rate constants for chemical reactions and phase partitioning. The computational model components added to track various chemical species are herein referred to as “tracers” because their computational presence is not necessarily used to adjust any hydraulic properties of the liquid phase or gas phase. The inventors have discovered that this is very useful for oxidation reactions, perhaps because the liquid phase concentrations of the various types of oxidizable compound are often under 10 weight percent and sometimes under 1 weight percent in the preponderance of the reaction medium. Optionally, the weight percent of solids is accounted in the computational model, largely in consideration of the feed plumes of solvent and of oxidizable compound feed, which are often lower in solids. The disclosed commercial CFD software packages are amenable in all these respects.

The computational model according to the present invention is thus capable of calculating the concentration of one or more species of oxidizable compound using reactive tracers over time within the entire bubble column reactor, divided into small sub-volumes according to the computational meshing. Furthermore, the computational model according to the present invention is capable of calculating the concentration of the gas phase of the reaction medium and of calculating the concentration of dissolved oxidant in the liquid phase of the reaction medium.

In step 236, the CFD model is run to stochastic quasi-steady-state to obtain transient and time-averaged calculations of chemical compositions throughout the bubble column. Importantly, even after the computational model has reached stochastic quasi-steady-state, the chemical concentrations of some chemical species will rise and fall significantly in various computational cells of the reaction medium from one time increment to the next. In fact, the total amount of some chemical species summed up throughout the entire reaction medium will rise and fall from one time increment to the next. This is owing to the chaotic flow patterns within the bubble column reaction medium. Thus, it is preferable to recognize the stochastic nature of the system by time-averaging the computational model calculations for chemical species through times lasting at least about 10 seconds, more preferably through times lasting at least about 100 seconds, and most preferably through times lasting between 100 seconds and 1,000 seconds.

In step 238, actual/measured chemical composition data at certain specified locations in an operating bubble column reactor are obtained. The reactor from which the actual/measured chemical composition data is taken preferably is configured and operated in accordance with the description provided above with reference to FIGS. 1-19. Whether using a bubble column reactor or another type reactor, the inventors have discovered that it is important to obtain actual/measured data for chemical composition from an oxidation reactor operating at appropriately similar conditions including, for example, type of oxidizable compound, STR, pressure, temperature, solvent composition, catalyst composition, and gradients in oxidizable compound, oxidant, and local oxygen-STR. These gradients are particularly difficult to simulate appropriately in laboratory-scale and pilot-scale equipment, and relevant data are lacking in the open literature. However, the inventors have discovered that these gradients are particularly important when obtaining appropriate measured data for chemical composition to use in validation of computational models. The inventors have also discovered that the reaction rates of para-tolualdehyde, para-toluic acid, and 4-CBA, as well as para-xylene, all exhibit fractional-order dependence of reaction rates on the liquid phase concentrations when the STR is pushed though a range greater than about two to one using the preferred embodiments disclosed herein. Furthermore, the present inventors have noted other peculiar dependencies of reaction rates when the reaction medium is operated with gradients in oxidizable compound and oxygen-STR according to embodiments of the present invention. Perhaps such dependencies again relate to a much higher, near second-order termination rate of free radicals in an intense reaction zone that is not completely offset by reduced, though still near second-order, termination rate in a less intense zone. In addition, in such a reaction medium with preferred gradients, the ratio of one reactant species to another varies widely from one location to another, even in a time-averaged sense. Whatever the underlying chemical causes, such dependencies are very difficult to determine experimentally in smaller vessels or in relatively well-mixed vessels where the ratio of various reacting species, including free radicals, is more uniform throughout the reaction medium.

Whatever the underlying chemical mechanisms, the inventors have discovered that obtaining measured data for chemical composition from an operating oxidation reactor is particularly pertinent when modeling oxidation reactors, including bubble columns, where spatial gradients exist for concentrations of some species of oxidizable compound, concentrations of oxidant, and oxygen-STR. Preferably, measured data for chemical composition is obtained for reaction medium operating at conditions appropriately similar to those being modeled, as now described.

Preferably, the measured data for chemical composition is obtained using a reaction medium wherein the water content is within 6 weight percent, more preferably 2 weight percent, most preferably 1 weight percent of the composition of the reaction medium being modeled. When acetic acid is used as solvent, the measured data for chemical composition is preferably obtained using a reaction medium wherein the acetic acid content is within 6 weight percent, more preferably 2 weight percent, most preferably 1 weight percent of the composition of the modeled reaction medium.

Preferably, the measured data for chemical composition is obtained using a reaction medium wherein the concentration of the individual components of the catalyst system are within 50 percent, more preferably 25 percent, most preferably 10 percent of the reaction medium being modeled. For example, if cobalt at 2,000 ppmw is one component of the catalyst system being modeled, then the ranges for obtaining measured data for chemical composition are 1,000 to 3,000 ppmw, 1,500 to 2,500, and 1,800 to 2,200, in order of preference.

Preferably, the measured data for chemical composition is obtained using a temperature at the mid-height of the reaction medium that is within about 32° C., more preferably about 16° C., still more preferably about 8° C., most preferably 4° C. of the temperature at the mid-height of the modeled reaction medium.

Preferably, the measured data for chemical composition is obtained using a pressure at the top of the reaction medium that is within about 0.4 megapascal, more preferably about 0.2 megapascal, still more preferably about 0.1 megapascal, most preferably 0.05 megapascal of the pressure at the top of the modeled reaction medium.

Preferably, the measured data for chemical composition is obtained using a STR that is within about 80 percent, more preferably about 40 percent, most preferably 20 percent of the modeled reaction medium. For example, if para-xylene is fed in the model at a STR of 50 kilograms per cubic meter of reaction medium per hour, then the ranges for obtaining measured data for chemical composition are, in order of preference, 10 to 90, 30 to 70, and 40 to 60 kilograms per cubic meter per hour.

Preferably, the measured data for chemical composition is obtained using an actual reaction medium wherein the ratio of the oxidizable compound concentration of the OC-max horizontal slice to the oxidizable compound concentration of the OC-min horizontal slice is greater than about 5:1, more preferably greater than about 10:1, still more preferably greater than about 20:1, and most preferably in the range of from 40:1 to 1,000:1.

Preferably, the measured data for chemical composition is obtained using an actual reaction medium wherein the ratio of the oxygen-STR of a first distinct 20-percent continuous volume of the reaction medium compared to the oxygen-STR of a second distinct 20-percent continuous volume of the reaction medium is in the grange of from about 1.5:1 to about 20:1, more preferably in the range of from about 2:1 to about 12:1, and most preferably in the range of from 3:1 to 9:1.

Preferably, the measured data for chemical composition is obtained using an actual reaction medium wherein the ratio of the partial pressure of molecular oxygen at the gas outlet(s), which is often the top of the reaction medium, compared to the partial pressure of molecular oxygen at the corresponding position of the modeled reaction medium is in the range of from about 0.4:1 to about 20:1, more preferably in the range of from about 0.8:1 to about 4:1, and most preferably in the range of from 0.9:1 to 1.4:1. Preferably, the measured data for chemical composition is obtained using an actual reaction medium wherein the ratio of the partial pressure of molecular oxygen at the oxidant inlet(s) compared to the partial pressure of molecular oxygen at the corresponding position of the modeled reaction medium is in the range of from about 0.4:1 to about 20:1, more preferably in the range of from about 0.8:1 to about 4:1, and most preferably in the range of from 0.9:1 to 1.4:1.

Preferably, the measured data for chemical composition is obtained using an actual reaction medium wherein the ratio of the average gas hold-up compared to the average gas hold-up of the modeled reaction medium is in the range of from about 0.2:1 to about 2:1, more preferably in the range of from about 0.5:1 to about 1.6:1, and most preferably in the range of from 0.8:1 to 1.3:1.

If precipitated solids are present, it is preferable that the measured data for chemical composition is obtained with a solids content of above about 4 weight percent of total slurry weight, more preferably between about 8 weight percent and about 45 weight percent of total slurry weight, and most preferably between 15 weight percent and 35 weight percent of total slurry weight. Preferably, the measured data for gas hold-up is obtained with the solids content in the slurry within about 15 weight percent of the intended modeling condition, more preferably within about 10 weight percent of the intended modeling condition, and most preferably within 3 weight percent of the intended modeling condition. For example, if the model target is 31 weight percent solids in the slurry, then the most preferable range for obtaining measured data for gas hold-up is 28 to 34 weight percent solids.

Preferably, the measured data for chemical composition is obtained with the maximum width of the actual reaction medium in excess of about 0.2 meters, more preferably between about 1 and about 15 meters, and most preferably between 2 and 10 meters. Preferably, the measured data for chemical composition is obtained with the depth of the actual reaction medium in excess of about 0.5 meters, more preferably between about 2 and about 90 meters, and most preferably between 5 and 50 meters. Preferably, the measured data for gas hold-up is obtained with the H:W ratio of the actual reaction medium in the range of from about 2:1 to about 30:1, still more preferably in the range of from about 3:1 to about 20:1, and most preferably in the range of from 4:1 to 12:1.

Preferably, measured data for chemical composition is obtained for the concentration of various chemical species for at least one vertical position within the actual reaction medium, more preferably for at least two vertical positions separated by at least 10 percent of the total height of the reaction medium, and still more preferably for at least three vertical positions comprising at least 50 percent of the total height of the reaction medium.

Preferably, measured data for chemical composition is obtained for the concentration of various chemical species for at least one radial position within the actual reaction medium, more preferably for at least two radial positions separated by at least 10 percent of the maximum diameter of the reaction medium, and still more preferably for at least three radial positions comprising at least 50 percent of the maximum diameter of the reaction medium.

When some aspect of the reactor, such as the feed of oxidizable compound, is not symmetric azimuthally, measured data for chemical composition is preferably obtained for the concentration of various chemical species for at least two azimuthal positions within the reaction medium separated by at least 45 degrees of angular rotation, and more preferably for at least three azimuthal positions having at least 90 degrees of angular rotation.

Preferably, measured data for chemical composition is collected from each location enough times to obtain the time-averaged concentrations at a each location, preferably at least 3 samples per location, more preferably at least 5 samples per location, and most preferably as determined necessary to know the mean value within 10 percent at a 95 percent confidence interval using statistical analysis.

In step 240, the CFD model calculations are compared to the actual/measured chemical composition data. In comparing the computational model calculations to measured data for chemical composition, it is preferable that the time-averaged modeled concentration for each relevant chemical species be within about 32 percent, more preferably about 16 percent, still more preferably about 8 percent, and most preferably 4 percent, of the time-averaged measured concentration for the respective chemical species at the respective position. For example, if the measured concentration of para-xylene at a particular position in the actual reaction medium is 500 ppmw, the most preferred range for the model concentration of para-xylene at that location in the modeled reaction medium is 480 to 520 ppmw.

In step 242, a decision is made as to whether the CFD model matches the actual chemical data well enough. If the various comparisons of the computational model to the measured data for chemical composition indicate acceptable agreement, then the vast additional output of the computational model is deemed useful for analysis and actions as described in disclosure further below. However, if there is insufficient agreement with the measured data for chemical composition, then adjustment of the configuration of the CFD model is indicated before re-running the model to obtain improved reconciliation with the measured data for chemical composition.

In accordance with step 244, if the differences between the modeled and actual chemical data are outside the desired ranges, it is preferable to adjust the reactive tracer chemistry model included in the computational model using a species-by-species review of chemical reaction algorithms and chemical reaction rate constants. For example, if the concentration of a particular species is consistently too low in all material phases compared to the measured data for chemical composition, then the algorithms for creation of the species must be adjusted to show faster creation and/or the algorithms for consumption of the species must be adjusted to show slower consumption. It is also preferable to conduct a species-by-species review of phase equilibrium algorithms and phase transfer rate algorithms and constants. For example, if the total amount of the species matches appropriately with the measured data for chemical composition but the partitioning is in error between solid, liquid or gaseous phases, then the phase transfer rate algorithms and constants are indicated for improvement. After adjustment of the chemistry model, the inventive method can return to step 236 to re-run the CFD program with the improved chemistry model.

Once computational model parameters have been adjusted to obtain fidelity with the measured data for chemical composition, the inventive method proceeds to step 246 for use of the model to revise existing reactor designs or design new reactors. The model output is especially useful to evaluate the initial dispersion of oxidizable compound feed into the reaction medium, to evaluate the gradients and staging of oxidizable compound and oxidant within the reaction medium, and to evaluate the dissolved oxygen concentrations throughout the reaction medium.

In step 248, various data generated by the model is compared to the desired data. The model data is inspected to ascertain whether mechanical and process modifications are appropriate to improve the reactor. For example, the model calculations can be used to analyze the dispersion of oxidizable compound. One preferred method for this analysis is to identify for each time increment the computational cells of reaction medium containing concentrations of oxidizable compound reactive tracer above certain thresholds within the liquid phase; and these computational cells are referred to herein as offending cells. The volumes of these offending cells of reaction medium are then added together to find the total volume of offending reaction medium at each time increment through a specified time interval. For ease of comparison to other design options, this total volume of offending reaction medium can be normalized, dividing by the total volume of the entire reaction medium. Optionally, and rather than summing using the entire volume of each offending cell, one can again use the same offending cells but sum only the volume or mass of the liquid, or slurry, within each cell. By addition, the volume or mass of all offending liquid, or slurry, is found; and these can be normalized by the total volume or mass of all liquid, or slurry, as appropriate. As a further option, identification thresholds can be set for offending cells based on the mass of oxidizable compound reactive tracer in a calculational cell without respect of how much liquid phase is in the cell. However, this is often a less desirable method when the preponderance of oxidation reaction takes places in the liquid phase, because the concentration of the various reactive species in the liquid phase is of greater importance to chemical reaction kinetics than is the concentration of reactive species in space. Yet another option for analyzing distribution of oxidizable compound feed involves determining the maximum and minimum volumes of offending cells occurring within a specified time interval. In the case of modeling a bubble column reactor, it is preferable to recognize the stochastic nature of the bubble column reactor by taking said time interval to be at least about 10 seconds, more preferably at least about 100 seconds, and most preferably between 100 and 1,000 seconds.

Dissolved oxygen concentration can be calculated throughout the reaction medium using the computational model of the present invention by including calculations of gas-liquid transfer rates, by summing most or all significant chemical demand for dissolved oxygen, and by accounting for oxygen remaining in the gas phase of each calculational cell for each time step. Taking the case of para-xylene feed as an example, reactive tracer species preferably include para-xylene, para-tolualdehyde, para-toluic acid, 4-CBA, dissolved molecular oxygen in the liquid phase, and molecular oxygen in the gas phase. Optionally, terephthaldehyde, 4-hydroxymethyl benzoic acid, and yet other reactive tracer species can be added in the liquid phase, and vaporized para-xylene reactive tracer can be added in the gas phase. The models provided for such reactive tracer species include for their flow into the reaction medium, their flow out, their creation within the reaction medium, and for their consumption. The total demand for dissolved oxygen reactive tracer is summed from the stoichiometry of each of the individual reactive tracers that consume oxygen.

In step 250, the decision is made as to whether certain operating parameters (e.g., oxidizable compound dispersion and dissolved oxygen concentration) are good enough. The modeling analysis of a particular reactor design is considered complete when the output of a reconciled computational model matches certain disclosed preferred conditions such as, for example, dispersion of oxidizable compound, gradients and staging of chemical compositions in gas and liquid phases, and space time reaction rates and their gradients.

In accordance with step 252, if the reconciled computational model indicates that some of the preferred conditions are not met, then modifications of the mechanical and/or process design are made in an attempt to improve one or more of the conditions. When modifications for the mechanical and/or process design are indicated, consideration is given to the various disclosed preferred design features disclosed herein. These provide objectives for chemical compositions and reaction rates within the reaction medium, including recognition of the spatial and temporal variation, along with mechanical methods for obtaining these objectives. For example, it may be useful to add more feed points or better positioned feed points for oxidizable compound or to increase the inlet velocity of oxidizable compound in order to improve its initial dispersion. For example, it may be useful to add upright surfaces or non-fouling baffles to adjust the end-to-end gradients of chemical composition, STR or oxygen STR. For example, it may be useful to adjust the various physical dimensions of a reactor design in order to raise or lower the global average STR or to change the superficial gas velocity and the attendant mixing and mass transfer characteristics. All of these and other means disclosed herein for optimal design of a bubble column oxidation reactor, plus those means separately known in the art, may be considered in various combinations for improving reactor performance; and the disclosed modeling method is repeated. Once the design has been modified in accordance with step 252, the inventive method returns to step 204.

In accordance with step 254, once CFD model parameters have been adjusted to obtain fidelity with the measured data for gas hold-up and for chemical composition, these CFD model parameters are useful to design completely new reactors with reaction medium at suitably similar ranges of various parameters, as disclosed herein. This is particularly relevant for oxidation bubble columns, because the flow patterns, mixing and chemistry of various competing, parallel and sequential reactions are all important to obtaining the appropriate levels of dissolved oxidant and the preferred balance of reaction selectivity as disclosed in other aspects of the current invention. Optionally, the CFD model can be used to study dynamic response to changes in process conditions (e.g., disturbances in pressure; disturbances in feed rates, locations and compositions of oxidant; disturbances in feed rates, locations and compositions of oxidizable compound; and/or disturbances in feed rates, locations and compositions of solvents).

The inventors note that for all numerical ranges provided herein, the upper and lower ends of the ranges can be independent of one another. For example, a numerical range of 10 to 100 means greater than 10 and/or less than 100. Thus, a range of 10 to 100 provides support for a claim limitation of greater than 10 (without the upper bound), a claim limitation of less than 100 (without the lower bound), as well as the full 10 to 100 range (with both upper and lower bounds).

The invention has been described in detail with particular reference to preferred embodiments thereof, but will be understood that variations and modifications can be effected within the spirit and scope of the invention.

We claim:

1. A process comprising:
(a) oxidizing an oxidizable compound in a liquid phase of an actual multi-phase reaction medium contained in an actual oxidation reactor;
(b) determining at least one measured gas hold-up value for said actual reaction medium based on actual measurements taken during said oxidizing of step (a); and
(c) generating a computer model of a modeled oxidation reactor containing a modeled reaction medium;
(d) using said computer model to determine at least one modeled gas hold-up value for said modeled reaction medium; and
(e) comparing said modeled and measured gas hold-up values to one another.

2. The process of claim 1 wherein step (e) includes comparing modeled and measured gas hold-up values that are time-averaged over at least about 10 seconds.

3. The process of claim 2 wherein step (e) includes comparing modeled and measured gas hold-up values that are volume-averages of two or more discrete volumes in each of said modeled and actual reaction mediums.

4. The process of claim 3 wherein said two or more discrete volumes are vertically and/or radially spaced from one another.

5. The process of claim 1 further comprising, determining whether said modeled gas hold-up value matches said measured gas hold-up value closely enough.

6. The process of claim 5 wherein said modeled and measured gas hold-up values match closely enough if said modeled gas hold-up value is within 0.9 to 1.1 times said measured gas hold-up value.

7. The process of claim 5 further comprising, adjusting one or more parameters of said computer model if said modeled gas hold-up value does not match said measured gas hold-up value closely enough.

8. The process of claim 7 further comprising, repeating step (d) with the adjusted model parameters.

9. The process of claim 1 wherein said actual measurements are taken by emitting radiation on one side of said actual reactor, causing the radiation to travel through a portion of said actual reaction medium, and detecting the radiation one the other side of said actual reactor.

10. The process of claim 1 wherein said actual measurements are obtained using computed tomography (CT) scanning.

11. The process of claim 1 wherein said actual measurements include at least one horizontal, cross-section gas hold-up profile of said actual reaction medium.

12. The process of claim 1 wherein said computer model employs computational fluid dynamics (CFD) modeling.

13. The process of claim 1 wherein said computer model includes a physical model and a chemistry model.

14. The process of claim 1 further comprising, determining at least one measured reactant concentration value of said actual reaction medium based on actual measurements taken during said oxidizing of step (a), using said computer model to determine at least one modeled reactant concentration value for said modeled reaction medium, and comparing said modeled and measured reactant concentration values to one another.

15. The process of claim 14 further comprising, determining whether said modeled reactant concentration value matches said measured reactant concentration value closely enough.

16. The process of claim 15 wherein said modeled and measured reactant concentration values match closely enough if said modeled reactant concentration value is within about 32 percent of said measured reactant concentration value.

17. The process of claim 14 wherein said measured and modeled reactant concentration values include para-xylene concentration and/or oxygen concentration.

18. The process of claim 1 wherein said oxidizable compound is an aromatic compound.

19. The process of claim 1 wherein said oxidizable compound is para-xylene

20. The process of claim 19 wherein said actual oxidation reactor is a bubble column reactor.

21. The process of claim 20 wherein said actual reaction medium has a maximum width (W) of at least about 0.2 meters, a maximum height (H) of at least about 0.5 meters, and an H:W ratio of at least about 2:1.

22. The process of claim 20 wherein said actual reaction medium has a maximum width (W) of at least 2 meters, a maximum height (H) of at least 5 meters, and an H:W ratio of at least 4:1.

23. The process of claim 20 wherein said actual reaction medium has a solids content of at least about 4 percent by weight.

24. A process comprising:
(a) oxidizing an oxidizable compound in a liquid phase of an actual multi-phase reaction medium contained in an actual oxidation reactor;
(b) determining at least one measured reactant concentration value of said actual reaction medium based on actual measurements taken during said oxidizing of step (a);
(c) generating a computer model of a modeled oxidation reactor containing a modeled reaction medium;
(d) using said computer model to determine at least one modeled reactant concentration value for said modeled reaction medium; and
(e) comparing said modeled and measured reactant concentration values to one another.

25. The process of claim 24 wherein step (e) includes comparing modeled and measured reactant concentration values that are time-averaged over at least about 10 seconds.

26. The process of claim 24 wherein step (e) includes comparing modeled and measured reactant concentration values from two or more discrete locations in each of said actual and measured reaction mediums.

27. The process of claim 26 wherein said two or more discrete locations are vertically and/or radially spaced from one another.

28. The process of claim 24 further comprising, determining whether said modeled reactant concentration value matches said measured reactant concentration value closely enough.

29. The process of claim 28 wherein said modeled and measured reactant concentration value match closely enough if said modeled reactant concentration value is within about 32 percent of said measured reactant concentration value.

30. The process of claim 28 further comprising, adjusting one or more parameters of said computer model if said modeled reactant concentration value does not match said measured reactant concentration value closely enough.

31. The process of claim 30 further comprising, repeating step (d) with the adjusted model parameters.

32. The process of claim 24 wherein said measured and modeled reactant concentration values include oxygen concentration and/or oxidizable compound concentration.

33. The process of claim 24 wherein said computer model employs computational fluid dynamics (CFD) modeling.

34. The process of claim 24 wherein said computer model includes a physical model and a chemistry model.

35. The process of claim 24 further comprising, determining at least one measured gas hold-up value of said actual reaction medium based on actual measurements taken during said oxidizing of step (a), using said computer model to determine at least one modeled gas hold-up value for said modeled reaction medium, and comparing said modeled and measured gas hold-up values to one another.

36. The process of claim 24 wherein said oxidizable compound is an aromatic compound.

37. The process of claim 24 wherein said oxidizable compound is para-xylene

38. The process of claim 37 wherein said actual oxidation reactor is a bubble column reactor.

39. The process of claim 38 wherein said actual reaction medium has a maximum width (W) of at least about 0.2 meters, a maximum height (H) of at least about 0.5 meters, and an H:W ratio of at least about 2:1.

40. The process of claim 38 wherein said actual reaction medium has a maximum width (W) of at least 2 meters, a maximum height (H) of at least 5 meters, and an H:W ratio of at least 4:1.

41. The process of claim 38 wherein said actual reaction medium has a solids content of at least about 4 percent by weight.

42. A process comprising:
(a) oxidizing para-xylene in a liquid phase of an actual multi-phase reaction medium contained in an actual bubble column reactor;
(b) determining at least one measured gas hold-up value and at least one measured reactant concentration value for said actual reaction medium based on actual measurements taken during said oxidizing of step (a);
(c) generating a computer model of a modeled bubble column oxidation reactor containing a modeled multi-phase reaction medium;
(d) using said computer model to determine at least one modeled gas hold-up value and at least one modeled reactant concentration value for said modeled reaction medium; and
(e) adjusting one or more parameters of said computer model based on a comparison of said measured and modeled gas hold-up values and/or a comparison of said measured and modeled reactant concentration values.

43. The process of claim 42 wherein said measured and modeled gas hold-up and reactant concentration values are time-averaged over at least about 10 seconds.

44. The process of claim 43 wherein said measured and modeled gas hold-up values include values that are volume-averaged over the entire volume of said actual and modeled reaction mediums.

45. The process of claim 44 wherein said measured and modeled gas hold-up values include at least two volume-averaged values for corresponding vertically-spaced locations in said actual and modeled reaction mediums.

46. The process of claim 43 wherein said measured and modeled reactant concentration values include at least two values at corresponding vertically-spaced locations in said actual and modeled reaction mediums.

47. The process of claim 46 wherein said measured and modeled reactant concentration values include at least two values at corresponding radially-spaced locations in said actual and modeled reaction mediums.

48. The process of claim 42 further comprising, repeating step (d) with the adjusted model parameters.

49. The process of claim 42 wherein said measured and modeled reactant concentration values include para-xylene and/or oxygen concentration.

50. The process of claim 42 wherein said reaction medium has a maximum width (W) of at least 2 meters, a maximum height (H) of at least 5 meters, and an H:W ratio of at least 4:1, wherein said actual reaction medium has a solids content of at least about 4 percent by weight.
